SEQUENCE LISTING (42 sequences)

SEQ ID NO: 1 - DNA, 36 bp, synthetic construct
  source 1..36: mol_type = other DNA
actcgagctctgtagttgatttgacagttgacatcc

SEQ ID NO: 2 - DNA, 41 bp, synthetic construct
  source 1..41: mol_type = other DNA
ctaacccgggcactatattatcagtaagtggttgataaacc

SEQ ID NO: 3 - DNA, 26 bp, synthetic construct
  source 1..26: mol_type = other DNA
ctaacccgggcgtgttgatgtcctgc

SEQ ID NO: 4 - DNA, 31 bp, synthetic construct
  source 1..31: mol_type = other DNA
acgcgtcgacagttcagttcaggctgtacgc

SEQ ID NO: 5 - DNA, 34 bp, synthetic construct
  source 1..34: mol_type = other DNA
ctaacccgggcaagcaactctatgtcggtggaat

SEQ ID NO: 6 - DNA, 29 bp, synthetic construct
  source 1..29: mol_type = other DNA
tatcaagcttggcaaggccaatacacagc

SEQ ID NO: 7 - DNA, 35 bp, synthetic construct
  source 1..35: mol_type = other DNA
tatcaagcttagggttagagtgtctcgtttttgta

SEQ ID NO: 8 - DNA, 28 bp, synthetic construct
  source 1..28: mol_type = other DNA
ctaacccgggccagaccagaactattcc

SEQ ID NO: 9 - DNA, 20 bp, synthetic construct
  source 1..20: mol_type = other DNA
tctggaatcgaactctctcg

SEQ ID NO: 10 - DNA, 42 bp, synthetic construct
  source 1..42: mol_type = other DNA
attttgagacacaacgtggctttcatggcttaccccttgttg

SEQ ID NO: 11 - DNA, 39 bp, synthetic construct
  source 1..39: mol_type = other DNA
ttcacgaggcagacctcataaacatctgcgtttcccttg

SEQ ID NO: 12 - DNA, 20 bp, synthetic construct
  source 1..20: mol_type = other DNA
ttgcttctgctttaactcgg

SEQ ID NO: 13 - DNA, 23 bp, synthetic construct
  source 1..23: mol_type = other DNA
atgagccatattcaacgggaaac

SEQ ID NO: 14 - DNA, 25 bp, synthetic construct
  source 1..25: mol_type = other DNA
ttagaaaaactcatcgagcatcaaa

SEQ ID NO: 15 - DNA, 36 bp, synthetic construct; identical to SEQ ID NO: 1
  source 1..36: mol_type = other DNA

SEQ ID NO: 16 - DNA, 41 bp, synthetic construct; identical to SEQ ID NO: 2
  source 1..41: mol_type = other DNA

SEQ ID NO: 17 - DNA, 26 bp, synthetic construct
  source 1..26: mol_type = other DNA
ctaacccgggcgtgttgatgtcctgc

SEQ ID NO: 18 - DNA, 31 bp, synthetic construct; identical to SEQ ID NO: 4
  source 1..31: mol_type = other DNA

SEQ ID NO: 19 - DNA, 34 bp, synthetic construct; identical to SEQ ID NO: 5
  source 1..34: mol_type = other DNA

SEQ ID NO: 20 - DNA, 29 bp, synthetic construct
  source 1..29: mol_type = other DNA
tatcaagcttggcaaggccaatacacagc

SEQ ID NO: 21 - DNA, 35 bp, synthetic construct; identical to SEQ ID NO: 7
  source 1..35: mol_type = other DNA

SEQ ID NO: 22 - DNA, 28 bp, synthetic construct
  source 1..28: mol_type = other DNA
ctaacccgggccagaccagaactattcc

SEQ ID NO: 23 - DNA, 30 bp, synthetic construct
  source 1..30: mol_type = other DNA
ctagtctagaagtgctttcagtgggtgacg

SEQ ID NO: 24 - DNA, 31 bp, synthetic construct
  source 1..31: mol_type = other DNA
agcttgatatcccatgcttttccagtttcgg

SEQ ID NO: 25 - DNA, 32 bp, synthetic construct
  source 1..32: mol_type = other DNA
agcttgatatcggcgaaatccaaccgtataag

SEQ ID NO: 26 - DNA, 30 bp, synthetic construct
  source 1..30: mol_type = other DNA
ccgctcgagggggaagttgttaagacagac

SEQ ID NO: 27 - DNA, 35 bp, synthetic construct
  source 1..35: mol_type = other DNA
agcttgatatcagagtgtgttgatagtgcagtatc

SEQ ID NO: 28 - DNA, 34 bp, synthetic construct
  source 1..34: mol_type = other DNA
agcttgatatcacctctttagcttcttggaagct

SEQ ID NO: 29 - DNA, 70 bp, synthetic construct
  source 1..70: mol_type = other DNA
taaaatattaatgatgattatggtaggggcattcgcactaaataatgaaagtgtaggctggagctgcttc

SEQ ID NO: 30 - DNA, 70 bp, synthetic construct
  source 1..70: mol_type = other DNA
acaactagtggaaatacctgtactttataatttcaagggtacgggtccgccatatgaatatcctccttag

SEQ ID NO: 31 - DNA, 36 bp, synthetic construct
  source 1..36: mol_type = other DNA
ctagtctagaaacccgggtgatagtgtagcggcaca

SEQ ID NO: 32 - DNA, 27 bp, synthetic construct
  source 1..27: mol_type = other DNA
actcgagctcgtgagcaaagccagctg

SEQ ID NO: 33 - DNA, 27 bp, synthetic construct
  source 1..27: mol_type = other DNA
acgcgtcgacctcggtgtggaaattgg

SEQ ID NO: 34 - DNA, 29 bp, synthetic construct
  source 1..29: mol_type = other DNA
ctaacccgggcaacgtacttactctaccg

SEQ ID NO: 35 - DNA, 31 bp, synthetic construct
  source 1..31: mol_type = other DNA
agcttgatatctgtgacggaagatcacttcg

SEQ ID NO: 36 - DNA, 31 bp, synthetic construct
  source 1..31: mol_type = other DNA
agcttgatatcgggcaccaataactgcctta

SEQ ID NO: 37 - DNA, 70 bp, synthetic construct
  source 1..70: mol_type = other DNA
accgccaggcgtttaccgttagcgagagcaacaaggggtaagccatggccgtgtaggctggagctgcttc

SEQ ID NO: 38 - DNA, 70 bp, synthetic construct
  source 1..70: mol_type = other DNA
acccgctctctttcaagcaagggaaacgcagatgtttagataggctgcgtcatatgaatatcctccttag

SEQ ID NO: 39 - DNA, 70 bp, synthetic construct
  source 1..70: mol_type = other DNA
actatcaccagattgatttttgccttatccgaaactggaaaagcatggaagtgtaggctggagctgcttc

SEQ ID NO: 40 - DNA, 70 bp, synthetic construct
  source 1..70: mol_type = other DNA
ttttatttgatgggataaagatctttgcgcttatacggctggatttcgcccatatgaatatcctccttag

SEQ ID NO: 41 - DNA, 70 bp, synthetic construct
  source 1..70: mol_type = other DNA
cgccacccgcgctactggggagcatggctgggtgtagcagcgatggcggggtgtaggctggagctgcttc

SEQ ID NO: 42 - DNA, 70 bp, synthetic construct
  source 1..70: mol_type = other DNA
gcgagttttcagcaattttagtatccaggtgtattgttctggtcgcggaccatatgaatatcctccttag

(19) United States
(12) Patent Application Publication
(10) Pub. No.: US 2024/0358810 A1
(43) Pub. Date: Oct. 31, 2024

(54) Shigella Vaccine

(71) Applicant: GLAXOSMITHKLINE BIOLOGICALS S.A., Rixensart (BE)

(72) Inventor: Francesca Micoli, Siena (IT)

(21) Appl. No.: 18/686,089

(22) PCT Filed: Aug. 23, 2022
(86) PCT No.: PCT/EP2022/073501

§ 371 Date: Feb. 23, 2024

(30) Foreign Application Priority Data
Aug. 24, 2021 (GB) 2112149.6

(51) Int. Cl. A61K 39/112; A61K 39/00; A61P 31/04

(52) U.S. Cl. CPC A61K 39/0283; A61P 31/04; A61K 2039/545; A61K 2039/55505; A61K 2039/55572; A61K 2039/575

(57) ABSTRACT

The present invention relates to immunogenic compositions and their use in providing protection against illness caused by infection with Shigella. The immunogenic compositions comprise Shigella GMMA with particular doses of O-antigen.

### FIELD OF THE INVENTION

The present invention relates to immunogenic compositions and their use in providing protection against illness caused by infection with Shigella.

### BACKGROUND TO THE INVENTION

Shigellosis is a major global health problem, responsible for more than 7 million Disability-Adjusted Life Years and 100,000 deaths per year, especially in children under 5 years old in developing countries. Shigellosis is caused by Gram-negative bacteria of the genus Shigella, which is divided into 4 species and further differentiated into 50 serotypes based on the structure and composition of the outer polysaccharide antigen (O-antigen, OAg) of the lipopolysaccharide (LPS): S. sonnei (1 serotype), S. flexneri (15 serotypes), S. boydii (19 serotypes) and S. dysenteriae (15 serotypes). A limited number of serotypes contribute to the global burden of disease and these vary between regions and over time. Shigella sonnei and Shigella flexneri 2a are the currently dominant serotypes worldwide.

Previous Shigella vaccines have demonstrated low immunogenicity and lack of protection. Thus, it is an object of the invention to provide improved immunogenic compositions, particularly vaccine compositions that can be used to protect against multiple serotypes of Shigella. More particularly, it is an object to provide vaccine compositions that generate stronger responses to the O-antigen, especially in young children.

### SUMMARY OF THE INVENTION

The present application demonstrates that immunogenic compositions comprising GMMA with particular doses of O-antigen promote good levels of immunogenicity whilst being poorly reactogenic.

Accordingly, in the first aspect of the invention, there is provided an immunogenic composition comprising:
(a) Shigella sonnei GMMA; and
(b) Shigella flexneri GMMA,
   wherein each of the GMMA comprises modified lipid A, and the immunogenic composition comprises at least 10 μg of O-antigen.

In a second aspect of the invention, the invention provides an immunogenic composition of the invention for use in a method of preventing or treating infection by Shigella in a subject.

In a third aspect of the invention, there is provided a method of preventing or treating infection by Shigella in a subject comprising administering the immunogenic composition of the invention.

In a fourth aspect of the invention, the invention provides a use of the immunogenic composition of the invention in the manufacture of a medicament for use in a method of preventing or treating infection by Shigella in a subject.

### BRIEF DESCRIPTION OF THE FIGURES

FIG. 1. Percentage of participants with anti-S. sonnei LPS serum IgG≥268 EU/mL, geometric mean concentrations and geometric mean ratios by time point (per-protocol set).

FIG. 2. Anti-S. sonnei LPS serum IgG geometric mean concentrations (A) and geometric mean ratios (B) in participants who did or did not develop shigellosis post-challenge (per-protocol set). For each time period, from left to right, columns correspond to: 1790GAHB group (shigellosis); 1790GAHB group (no shigellosis); Placebo group (shigellosis); and Placebo group (no shigellosis).

LPS, lipopolysaccharide; IgG, immunoglobulin G; 1790GAHB/Placebo group (shigellosis), participants who received the 1790GAHB vaccine/placebo and developed shigellosis after the bacterial challenge; 1790GAHB/Placebo group (no shigellosis), participants who received the 1790GAHB vaccine/placebo and did not develop shigellosis after the bacterial challenge; GMC, geometric mean concentration; EU, enzyme-linked immunosorbent assay units; D1, baseline; D29, 28 days post-first 1790GAHB/placebo vaccination; D57, 28 days post-second 1790GAHB/placebo vaccination; D85, 28 days post-challenge dose; GMR, geometric mean ratio.

Note: Error bars depict 95% confidence intervals.
The upper limits of the 95% confidence intervals at D85 are 3,430.58 (1790GAHB group [shigellosis]) and 4,419·02 (Placebo group [shigellosis]) in panel A and 35.56 (Placebo group [shigellosis]) in panel B.
GMRs are calculated relative to D1 for D29 and D57, and relative to D57 for D85.

FIG. 3. Percentage of participants with anti-S. sonnei LPS serum IgG≥268 EU/mL, geometric mean concentrations and geometric mean ratios by time point (per-protocol set).

FIG. 4. Anti-S. sonnei LPS serum IgG antibody concentration at pre-challenge visit (D57) (per-protocol set). From left to right, box plots correspond to: 1790GAHB group (shigellosis); Placebo group (shigellosis); 1790GAHB group (no shigellosis); and Placebo group (no shigellosis).

FIG. 5. Vaccine efficacy of the 1790GAHB vaccine versus placebo against evaluated endpoints (per-protocol set).

FIG. 6. Grading of solicited adverse events

FIG. 7. Solicited adverse events 7 days post-vaccination (2 doses) (A) and 8 days post-challenge with S. sonnei (B) (solicited safety set).
N, number of participants in each group.
Note: Error bars depict 95% confidence intervals. Definitions of solicited adverse event grades are detailed in FIG. 6.

For both FIGS. 7A and 7B, for each adverse event, left-hand column corresponds to 1790GAHB group, and right-hand column corresponds to Placebo group. The wavy lines in some of the columns in FIG. 7A correspond to Grade 3, and the wavy lines in some of the columns in FIG. 7B correspond to Grade 3/4.

FIG. 8. Participants reporting unsolicited adverse events during the 28-day post-vaccination period (any dose) (unsolicited safety set)

FIG. 9. Participants reporting unsolicited adverse events during the 28-day post-challenge (unsolicited safety set)

FIG. 10. Anti-LPS/OAg IgG and SBA titres induced by altSonflex 1-2-3 in mice compared to corresponding monovalent formulations.

FIG. 11. Anti-LPS/OAg IgG induced by altSonflex 1-2-3 in rabbits compared to corresponding monovalent formulations

FIG. 12. SBA titers induced by altSonflex 1-2-3 in rabbits compared to corresponding monovalent formulations.

FIG. 13. Anti-LPS/OAg IgG levels induced by altSonflex 1-2-3 in mice when tested at different O-antigen doses.

FIG. 14. Anti-LPS/OAg IgG levels induced by altSonflex 1-2-3 in rabbits when tested at different O-antigen doses.

FIG. 15. SBA titers of antibodies elicited by altSonflex 1-2-3 in mice when tested at different O-antigen doses.

FIG. 16. SBA titers of antibodies elicited by altSonflex 1-2-3 in rabbits when tested at different O-antigen doses.

FIG. 17. Average temperature rise induced by altSonflex 1-2-3 toxicology lot and 4 component GMMA mix.

FIG. 18. altSonflex 1-2-3 toxicology lot induces similar proinflammatory cytokine release as 1790GAHB by MAT. Vaccine candidates were compared by starting dilutions from corresponding full human doses of 60 μg total OAg for altSonflex 1-2-3 and 6 μg OAg (100 μg protein) for 1790GAHB.

Line starting with a higher IL6 release at lower dilution time corresponds to altSonflex 1-2-3 and the line starting with the lower IL6 release at lower dilution time corresponds to 1790GAHB.

FIG. 19. altSonflex 1-2-3 leads to higher number of antibodies with stronger anti-S. sonnei bactericidal activity compared to 1790GAHB in human clinical trials. First four bars starting from the left corresponding to 1790GAHB studies. Last bar corresponds to altSonflex 1-2-3 study.

FIG. 20. altSonflex 1-2-3 induces antibodies with anti-S. flexneri 2a, 1b and 3a bactericidal activity.

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “An immunogenic composition comprising Shigella sonnei GMMA” should be interpreted to mean that the immunogenic composition contains Shigella sonnei GMMA, but the immunogenic composition may comprise further components. Similarly, the phrase “wherein the Shigella flexneri GMMA comprises Shigella flexneri serotype 1b GMMA” refers to Shigella flexneri GMMA that has Shigella flexneri serotype 1b GMMA, but the Shigella flexneri GMMA may contain GMMA of other Shigella flexneri serotypes.

In some embodiments of the invention, the word “comprising” is replaced with the phrase “consisting of”. The term “consisting of” is intended to be limiting. For example, the phrase “An immunogenic composition consisting of Shigella sonnei GMMA” should be understood to mean that the immunogenic composition has Shigella sonnei GMMA and no other components.

In some embodiments of the invention, the word “comprising” is replaced with the phrase “consisting essentially of”. The term “consisting essentially of” means that specific further components can be present, namely those not materially affecting the essential characteristics of the subject matter.

The term “about” or “around” when referring to a value refers to that value but within a reasonable degree of scientific error. Optionally, a value is “about x” if it is within 10%, within 5%, or within 1% of x. Similarly, if an amount of O-antigen in a Shigella flexneri serotype 1b GMMA is “about” 15 μg, the amount is within 10%, within 5%, or within 1% of 15 μg. Similarly, if a dose of immunogenic composition is administered around day 85, it may be administered at day 83, 84, 85, 86, or 87.

The singular forms “a”, “an”, and “the” include plural referents unless the content clearly dictates otherwise. Thus, for example, reference to “the GMMA” includes two or more instances or versions of such GMMA.

All publications, patents and patent applications cited herein, whether supra or infra, are hereby incorporated by reference in their entirety.

### Immunogenic Compositions

The present invention relates to immunogenic compositions comprising GMMA, specifically immunogenic compositions comprising Shigella sonnei GMMA and Shigella flexneri GMMA.

### Generalised Modules for Membrane Antigens (GMMA)

Generalised Modules for Membrane Antigens or GMMA are particles derived from the outer membrane of Gram-negative bacteria that have high levels of LPS, lipoproteins, proteins and other antigens that activate the innate immune response. GMMA are produced from genetically-modified bacterial strains that are mutated to enhance vesicle production and to remove or modify antigens (e.g. lipid A). Enhanced spontaneous generation of vesicles can be achieved, for example, by targeted deletion of proteins involved in maintenance of membrane integrity (see below). The outer surface of GMMA corresponds to the outer surface of the bacterium from which they are derived, preserving all membrane antigens (including e.g. lipopolysaccharides, lipooligosaccharides, lipoproteins, proteins) in the context of the membrane. GMMA (unlike detergent-extracted OMVs) retain these outer membrane components in their native conformation and correct orientation, better preserving immunogenicity against the bacterial strain from which they are derived. Thus, GMMA are highly immunogenic and this strong activation of innate immunity may lead to unacceptable reactions in human subjects, e.g. a febrile response or, in extreme cases, septic shock especially if parenterally administered.

The Shigella GMMA in the immunogenic compositions of the invention have a dose of O-antigen that makes the GMMA highly immunogenic but has a reduced risk of reactogenicity.

The term GMMA is used to provide a clear distinction from conventional detergent-extracted outer membrane vesicles (dOMV), and native outer membrane vesicles (NOMV), which are released spontaneously from Gram-negative bacteria. GMMA differ in two crucial aspects from NOMV. First, to induce GMMA formation, the membrane structure has been modified by the deletion of genes encoding key structural components, specifically tolR. Second, as a consequence of the genetic modification, large quantities of outer membrane “bud off” (the Italian word for bud is ‘gemma’) to provide a practical source of membrane material for vaccine production, leading to increased ease of manufacturing and potential cost reduction. While NOMV have been used for immunogenicity studies, the yields are too low for practical vaccines.

S. sonnei or S. flexneri GMMA used in the invention typically have a diameter of from 25 nm to 140 nm by electron microscopy, for example from 25 nm to 40 nm. GMMA may also have a bimodal size distribution. For example, the majority of GMMA having an average size from 25 nm to 40 nm in diameter (by EM) and a fraction of the particles having an average size from 65 nm to 140 nm. Particularly, at least 70%, at least 71%, at least 72%, at least 73%, at least 74%, at least 75%, at least 80%, at least 85%, at least 90% of the GMMA will have a diameter of from 25 nm to 140 nm.

GMMA-containing immunogenic compositions of the invention may be substantially free from whole bacteria, whether living or dead. The size of the GMMA means that they can readily be separated from whole bacteria by filtration e.g. as typically used for filter sterilisation. Although GMMA will pass through a standard 0.22 μm filters, these can rapidly become clogged by other material, and so it may be useful to perform sequential steps of filter sterilisation through a series of filters of decreasing pore size before using a 0.22 μm filter. Examples of preceding filters would be those with pore size of 0.8 μm, 0.45 μm, etc. GMMA are spontaneously-released from bacteria and separation from the culture medium, for example, using filtration, is convenient. Outer membrane vesicles formed by methods which involve deliberate disruption of the outer membrane (e.g. by detergent treatment, such as deoxycholate-extraction, or sonication) to cause outer membrane vesicles to form are excluded from the scope of the invention. GMMA used in the invention are substantially free from inner membrane and cytoplasmic contamination and contain lipids and proteins.

Shigella sonnei and Shigella flexneri GMMA

Shigella sonnei GMMA are GMMA from Shigella sonnei. Shigella sonnei GMMA are GMMA that have similar characteristics to GMMA that are produced by Shigella sonnei. In particular, Shigella sonnei GMMA comprise the Shigella sonnei O-antigen. In an embodiment, the Shigella sonnei GMMA are produced by and isolated from a strain of (i.e. derived from) Shigella sonnei. It is within the abilities of the skilled person to determine whether GMMA are Shigella sonnei GMMA. Specifically, the skilled person may use antibodies to detect whether the GMMA comprise the Shigella sonnei O-Antigen. Antibodies specific for different O-antigen are available. Dot blot analysis may be used to detect the binding of antibodies specific for different O-antigen. For example, a primary antibody may be applied to a sample of the GMMA, and binding of that primary antibody to the GMMA may be detected using a second antibody that is specific for the primary antibody and comprises a detection moiety such as an enzyme. A suitable dot blot analysis technique is described under the heading “OAg identity by dot blot” in Example 2. Alternatively, the presence of Shigella sonnei antigen may be determined using NMR, for example as discussed in Robbins et al. (PNAS (2009), 106:7974-7978).

Shigella flexneri GMMA are GMMA from a strain of Shigella flexneri. Shigella flexneri GMMA are GMMA that have similar characteristics to GMMA that are produced by Shigella flexneri. In particular, Shigella flexneri GMMA comprise a Shigella flexneri O-antigen. In an embodiment, the Shigella flexneri GMMA are produced by and isolated from a strain of (i.e. derived from) Shigella flexneri. It is within the abilities of the skilled person to determine whether GMMA are Shigella flexneri GMMA. Specifically, the skilled person may use antibodies to detect whether the GMMA comprise a Shigella flexneri O-Antigen. Antibodies specific for different O-antigen are available. Dot blot analysis may be used to detect the binding of antibodies specific for different O-antigen. For example, a primary antibody may be applied to a sample of the GMMA, and binding of that primary antibody to the GMMA may be detected using a second antibody that is specific for the primary antibody and comprises a detection moiety such as an enzyme. A suitable dot blot analysis technique is described under the heading “OAg identity by dot blot” in Example 2. Alternatively, the presence of Shigella sonnei antigen may be determined using NMR, for example as discussed in Perepelov et al. (FEMS Immunol. Med. Microbiol. (2012); 66:201-210).

Immunogenic compositions of the invention may comprise GMMA from at least two, three, four, five or six different strains of Shigella. The Shigella flexneri GMMA may be selected from the group consisting of serotype 1b GMMA, serotype 2a GMMA, serotype 3a GMMA and serotype 6 GMMA. The immunogenic compositions of the invention may comprise Shigella sonnei GMMA, Shigella flexneri serotype 1b GMMA, Shigella flexneri serotype 2a GMMA, and Shigella flexneri serotype 3a GMMA.

Immunogenic compositions of the invention include GMMA from more than one strain of Shigella and it is typical for the GMMA to be prepared separately prior to mixing them with pharmaceutically acceptable excipients, such as buffers. The GMMA from each strain may be individually formulated with an adjuvant, such as ALHYDROGEL®, prior to combining with GMMA from another strain and mixing with pharmaceutically acceptable excipient(s). Alternatively, the GMMA from each strain may be purified/isolated, combined with GMMA purified or isolated from the other strain(s), formulated with an adjuvant and then mixed with pharmaceutically acceptable excipient(s). Other methods will be apparent to one skilled in the art.

### Modification of Lipid A

The immunogenic compositions of the invention comprise GMMA that comprise modified lipid A and at least 10 μg of O-antigen. Particularly, such GMMA comprise modified lipid A that is less toxic compared to the corresponding wildtype lipid A.

“Toxicity” or “toxic” in this context refers to the extent to which the innate immune system is activated by lipid A, particularly through the Toll-like receptor 4 pathway. Highly toxic lipid A can lead to uncontrolled inflammation, apoptosis, and in extreme cases septic shock, among other effects. Therefore, a “less toxic” lipid A refers to a lipid A that activates the Toll-like receptor 4 pathway to a lesser extent relative to a corresponding wildtype lipid A, for example by having a structure that is less likely to be recognised by Toll-like receptor 4. Optionally, a modified lipid A is less toxic if it is less reactogenic than a corresponding wildtype lipid A. For example, one can determine whether a modified lipid A is less toxic by administering it to an animal such as a rabbit or mouse, and determine whether it activated more monocytes compared to a corresponding wildtype lipid A using the monocyte activation test described under the heading “Monocyte activation tests (MATs)” in the Examples.

“Corresponding wildtype lipid A” refers to lipid A that can be found in the corresponding wildtype bacterium and strain. For example, lipid A that is modified relative to a “corresponding wildtype lipid A” in the context of Shigella flexneri serotype 1b GMMA is interpreted to mean a modified lipid A (e.g. such that it is less toxic) relative to lipid A found in wildtype Shigella flexneri serotype 1b.

“LPS” refers to bacterial lipopolysaccharides.

In an embodiment, the Shigella GMMA of the invention are produced from Shigella strains that include one or more mutations resulting in inactivation of htrB, msbB1 and/or msbB2. Inactivation of htrB, msbB1 and msbB2 results in Shigella that produced GMMA comprising lipid A that is less toxic than wildtype lipid A.

By way of non-limiting example, suitable Shigella strains may be selected from the group consisting of ΔhtrB, ΔmsbB1 and ΔmsbB2 (ΔhtrB refers to a Shigella strain which has the htrB gene deleted). For simplicity, double deletions of both msbB1 and msbB2 may also be referred to as ΔDmsbB. Inactivation of htrB or msbB1 and msbB2 reduce acylation in lipid A. In the invention, inactivation of msbB1 and/or msbB2 is preferred.

“Inactivation” in the context of a gene refers to mutating or deleting the gene such that the protein to be transcribed can no longer carry out the function of the corresponding wildtype protein, or carries out the function to a lesser extent. For example, “inactivation of htrB” refers to deleting the gene, or mutating the gene, in a Shigella bacterium such that acylation of lipid A in the Shigella bacterium is reduced. Optionally, whether or not Shigella bacteria include one or mutations resulting in “inactivation” of htrB, or an msbB protein may be determined by isolating GMMA from the Shigella bacteria and analysing the lipid A in the GMMA using MALDI-TOF analysis. For example, one may use the technique described under the heading “Analysis of the lipid A by MALDI-TOF” in the Examples. One may compare the spectrum generated by MALDI-TOF analysis with a spectrum produced by analysing lipid A from Shigella that comprise wild type htrB and msbB genes, and if the amount of hexa-acylation is reduced, then the Shigella bacteria includes one or mutations resulting in inactivation of htrB and/or msbB.

Preferably the modified lipid A has a reduced level of hexa-acylated lipid A. GMMA for use in the immunogenic compositions of the invention may be from Shigella in which the virulence plasmid is lost. Loss of the virulence plasmid leads to loss of the msbB2 gene, and the chromosomal msbB/gene can be inactivated, thereby removing myristoyl transferase activity and providing a penta-acylated lipid A in the LPS. GMMA for use in the immunogenic compositions of the invention may be from S. flexneri msbB mutants lacking the virulence plasmid which contains the msbB2 gene. The Shigella GMMA of the immunogenic compositions of the invention are preferably from Shigella strains that express penta-acylated LPS. Alternatively, inactivation of htrB results in loss of the lauroyl chain and thus can yield penta-acylated LPS in some strains and/or forms of lipid A that are less toxic than wild type lipid A. For example, in S. flexneri, inactivation of htrB may be compensated for by the activity of another enzyme, LpxP that results in hexa-acylated lipid A, wherein the lauroyl-chain is replaced by a palmitoleoyl chain. Hexy-acylated lipid A comprising palmitoleoyl chains is less toxic than wild type lipid A. Thus, in some embodiments, the invention provides an immunogenic composition comprising Shigella sonnei and Shigella flexneri GMMA wherein the GMMA comprise penta-acylated lipid A and or hexa-acylated lipid A wherein the lauroyl-chain is replaced by a palmitoleoyl chain or wherein C14 comprises a myristoyl group. Suitable strains which the GMMA of the immunogenic compositions of the invention may be from include the following mutations (a) Shigella sonnei: ΔtolR, ΔhtrB, virG::nadAB, (b) Shigella flexneri 2a: ΔtolR, ΔmsbB, (c) Shigella flexneri 3a: ΔtolR, ΔmsbB and (d) Shigella flexneri 6: ΔtolR, ΔmsbB or ΔhtrB. Suitable strains are disclosed in the examples. Other suitable strains are known in the art, for example in WO2011/036564.

Optionally, the immunogenic composition of the invention comprises GMMA from Shigella sonnei that comprises mutations that inactivate msbB. Optionally the immunogenic composition of the invention comprises GMMA from Shigella sonnei that is ΔmsbB. Optionally, the immunogenic composition of the invention comprises GMMA from Shigella sonnei that is penta-acylated lipid A.

Optionally, the immunogenic composition of the invention comprises GMMA from Shigella flexneri 1b that comprises mutations that inactivate msbB. Optionally the immunogenic composition of the invention comprises GMMA from Shigella flexneri 1b that is ΔmsbB. Optionally, the immunogenic composition of the invention comprises GMMA from Shigella flexneri 1b that is penta-acylated lipid A and/or lipid A in which C14 comprises a myristoyl group.

Optionally, the immunogenic composition of the invention comprises GMMA from Shigella flexneri 2a that comprises mutations that inactivate msbB. Optionally the immunogenic composition of the invention comprises GMMA from Shigella flexneri 2a that is ΔmsbB. Optionally, the immunogenic composition of the invention comprises GMMA from Shigella flexneri 2a that is penta-acylated lipid A and/or lipid A in which C14 comprises a myristoyl group.

Optionally, the immunogenic composition of the invention comprises GMMA from Shigella flexneri 3a that comprises mutations that inactivate msbB. Optionally the immunogenic composition of the invention comprises GMMA from Shigella flexneri 3a that is ΔmsbB. Optionally, the immunogenic composition of the invention comprises GMMA from Shigella flexneri 3a that is penta-acylated lipid A and/or lipid A in which C14 comprises a myristoyl group.

Optionally, the immunogenic composition of the invention comprises GMMA from Shigella sonnei, Shigella flexneri 1b, Shigella flexneri 2a, and Shigella flexneri 3a that comprises mutations that inactivate msbB. Optionally, the immunogenic composition of the invention comprises GMMA from Shigella sonnei that is penta-acylated lipid A, Shigella flexneri 1b that is penta-acylated lipid A and/or lipid A in which C14 comprises a myristoyl group, Shigella flexneri 2a that is penta-acylated lipid A and/or lipid A in which C14 comprises a myristoyl group and Shigella flexneri 3a that is penta-acylated lipid A and/or lipid A in which C14 comprises a myristoyl group.

### O-Antigen

The Shigella GMMA of the invention are from Shigella strains that produce LPS comprising the O-antigen. The O-antigen is a polysaccharide moiety of the LPS, and its composition differs according to the strain/serotype of Shigella. O-antigen is exposed on the outer surface of Shigella, and thus can be recognised by host antibodies.

Shigella from which the GMMA are Produced

The Shigella GMMA in the immunogenic compositions of the invention may be derived from Shigella strains that are, relative to their corresponding wild-type strains, hyperblebbing i.e. they release into their culture medium larger quantities of GMMA than the corresponding wild-type strain. These GMMA are useful as components of Shigella vaccines of the invention. For example, the Shigella GMMA in the immunogenic compositions of the invention may be from a Shigella strain that is ΔtolR, or comprises a mutation that results in inactivation of tolR. Optionally, the Shigella sonnei GMMA is from a strain of Shigella sonnei that is ΔtolR. Optionally the Shigella flexneri GMMA is from at least one strain of Shigella flexneri that is ΔtolR.

GMMA are released spontaneously during bacterial growth and can be purified from the culture medium. The purification ideally involves separating the GMMA from living and/or intact Shigella bacteria, for example, by size-based filtration using a filter, such as a 0.2 μm filter, which allows the GMMA to pass through but which does not allow intact bacteria to pass through, or by using low speed centrifugation to pellet cells while leaving GMMA in suspension. Suitable purification methods are known in the art. A preferred two-step filtration purification process is described in WO 2011/036562 herein incorporated by reference. Particularly the two-step filtration process is used to separate GMMA from cell culture biomass without using centrifugation.

GMMA derived from a strain of Shigella may be produced by and purified from the Shigella strain, for example by using the methods described in the paragraph above.

### Dose of the Immunogenic Composition

The immunogenic compositions of the invention comprise at least 10 μg of O-antigen. Optionally, the immunogenic compositions may comprise any suitable amount of GMMA. Optionally, the immunogenic compositions of the invention correspond to a single dose. The term “dose” refers to an amount of pharmaceutical active, for example an amount of O-antigen, suitable for administration in one single dose, according to sound medical practice.

The amount of GMMA may be quantified by the amount of O-antigen in the GMMA. The immunogenic compositions of the invention may comprise, for GMMA of each strain included in the immunogenic composition, an amount of at least 10, 11, 12, 13, 14 or 15 μg of O-antigen. The amount of O-antigen may be about 15 μg. The amount of O-antigen may be between 10 μg and 25 μg, or between 12 μg and 20 μg. Therefore, for example, for an immunogenic composition that comprises Shigella sonnei GMMA, the amount of Shigella sonnei GMMA in the immunogenic compositions of the invention may be any of these. For an immunogenic composition that comprises Shigella flexneri serotype 1b, 2a, 3a or 6 GMMA, the amount of each of Shigella flexneri serotype 1b, 2a, 3a or 6 GMMA in the immunogenic composition of the invention may be any of these amounts.

The immunogenic compositions of the invention may comprise, for the total amount of GMMA from all strains included in the immunogenic composition, a total amount of at least 30, 35, 40, 45, 50, 55 or 60 μg of O-antigen. The total amount of O-antigen may be about 60 μg. The total amount of O-antigen may be between 40 μg and 100 μg, or between 50 μg and 70 μg. Therefore, for example, for an immunogenic composition that comprises Shigella sonnei GMMA and Shigella flexneri 1b, 2a, 3a and 6 GMMA, the total amount of O-antigen of GMMA from all strains may any of these amounts. The amount of O-antigen in GMMA from S. sonnei may be determined using HPAEC-PAD. For example, one may use the assay described in the Examples using the heading “OAg quantification by HPAEC-PAD (S. sonnei)”. The amount of O-antigen in GMMA from S. flexneri may be determined by the Hestrin-Dische assays. For example, one may use the assay described in the Examples under the heading “OAg O-acetyl content (S. flexneri 1b, 3a) by Hestrin-Dische”.

The amount of GMMA can also be quantified by measuring the amount of GMMA protein. GMMA protein may be from 0.1 to 200 μg per unit dose, particularly 10 μg, 20 μg, 25 μg, 50 μg or 100 μg. Per unit dose, aqueous immunogenic compositions of the invention may comprise a total concentration of GMMA protein of less than 200 μg/ml, less than 100 μg/ml or less, 80 μg/ml or less, 50 μg/ml or less, 25 μg/ml or less, 20 μg/ml or less, 15 μg/ml or less, 10 μg/ml or less. Per dose, aqueous immunogenic compositions of the invention may comprise a total concentration of GMMA protein of from 5 μg/ml to 200 μg/ml, from 5 μg/ml to 100 μg/ml, from 10 μg/ml to 100 μg/ml, from 10 μg/ml to 80 μg/ml, from 10 μg/ml to 50 μg/ml, 25 μg/ml to 50 μg/ml. Per dose, immunogenic compositions of the invention may comprise a total concentration of GMMA protein of more than 100 μg/ml, more than 80 μg/ml, more than 50 μg/ml, more than 25 μg/ml, more than 20 μg/ml, more than 15 μg/ml or more than 10 μg/ml.

GMMA protein from each different serotype may be present at an amount from 0.1 to 200 μg, for example from 0.1 to 80 μg, 0.1 to 100 μg and in particular from 5 to 25 μg. Suitable amounts of GMMA from each different serotype may include 0.1, 1, 5, 10, 20, 25, 30, 35, 40, 45, 50, 60, 70, 80, 90 and 100 μg per unit dose.

### Adjuvants

Immunogenic compositions of the invention may include one or more adjuvants. Particular adjuvants include aluminium adjuvants, for example, aluminium hydroxide (such as ALHYDROGEL®), aluminium phosphate, potassium aluminium sulphate and alum. The use of other adjuvants that also reduce the pyrogenic response is also envisaged and could be identified by the skilled person using the tests exemplified below. Whilst the term “adjuvant” generally refers to any substance that increases the immune response to an antigen, in the present case, and without wishing to be bound by hypotheses, the adjuvant, such as ALHYDROGEL®, is also an adsorbant reducing the immune response to the GMMA. Thus, the term “adsorbant” refers to a solid substrate or material to which the GMMA may bind, attach or adsorb (for example, by Van der Waals interactions or hydrogen bonding) such that the pyrogenic response to GMMA is reduced in comparison to GMMA that are not so bound, attached or adsorbed. By way of non-limiting example, immunogenicity of GMMA may be measured by comparing anti-LPS antibody response.

### Pharmaceutical Compositions (Immunogenic Compositions of the Invention Further Comprising a Pharmaceutically Acceptable Carrier)

The immunogenic compositions of the invention may further comprise a pharmaceutically acceptable carrier. Typical ‘pharmaceutically acceptable carriers” include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. Immunogenic compositions of the invention may also contain diluents, such as water, saline, glycerol, etc. Additionally, auxiliary substances, such as wetting or emulsifying agents, pH buffering substances, and the like, may be present. Sterile pyrogen-free, Tris-buffered physiologic saline is a preferred carrier particularly when using aluminium adjuvants since the phosphate in phosphate buffered saline may interfere with GMMA binding to aluminium.

Immunogenic compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The immunogenic composition may be prepared for topical administration e.g. as an ointment, cream or powder. The immunogenic composition may be prepared for oral administration e.g. as a tablet or capsule, as a spray, or as a syrup (optionally flavoured). The immunogenic composition may be prepared for pulmonary administration e.g. as an inhaler, using a fine powder or a spray. The composition may be prepared as a suppository or pessary. The immunogenic composition may be prepared for nasal, aural or ocular administration e.g. as drops. The immunogenic composition may be in kit form, designed such that a combined composition is reconstituted just prior to administration to a mammal. Such kits may comprise one or more antigens in liquid form and one or more lyophilised antigens. Immunogenic compositions may be presented in vials, or they may be presented in pre-filled syringes. The syringes may be supplied with or without needles. A syringe will include a single dose of the composition, whereas a vial may include a single dose or multiple doses.

Aqueous immunogenic compositions of the invention are also suitable for reconstituting other vaccines from a lyophilised form. Where an immunogenic composition of the invention is to be used for such extemporaneous reconstitution, the invention provides a kit, which may comprise two vials, or may comprise one ready-filled syringe and one vial, with the contents of the syringe being used to reactivate the contents of the vial prior to injection.

Immunogenic compositions of the invention may be packaged in unit dose form or in multiple dose form. For multiple dose forms, vials are preferred to pre-filled syringes.

Effective dosage volumes can be routinely established, but a typical human dose of the composition has a volume of 0.5 ml e.g. for intramuscular injection.

The pH of the immunogenic composition is preferably between 6 and 8, for example, 6, 6.5, 7, 7.5 or 8. For compositions comprising acetylated O-antigens particularly the pH of the composition is less than 7, preferably about 6 (to slow the rate of de-esterification). Stable pH may be maintained by the use of a buffer. The immunogenic compositions of the invention may comprise a Tris [Tris(hydroxymethyl)aminomethane] buffer. The Tris buffer may comprise about 1-20 mM [Tris(hydroxymethyl)aminomethane], e.g. 1.25 mM, 2.5 mM, 5.0 mM or 10.0 mM. For immunogenic compositions comprising acetylated O-antigens, in particular, the buffer is not a Tris buffer. The immunogenic compositions of the invention may comprise a 5-20 mM succinate buffer, e.g. 5 mM, 7.5 mM, 10 mM, 12.5 mM, 15 mM, 17.5 mM or 20 mM. The immunogenic compositions of the invention may comprise a 5-20 mM histidine buffer, e.g. 5 mM, 7.5 mM, 10 mM, 12.5 mM, 15 mM, 17.5 mM or 20 mM. The immunogenic compositions of the invention preferably comprise a 5-20 mM sodium phosphate buffer e.g. 5 mM, 7.5 mM, 10 mM, 12.5 mM, 15 mM, 17.5 mM or 20 mM. The composition will be sterile. Immunogenic compositions of the invention may be isotonic with respect to humans.

Thus, immunogenic compositions of the invention may be useful as vaccines. Vaccines according to the invention may either be prophylactic (i.e. to prevent infection) or therapeutic (i.e. to treat infection), but will typically be prophylactic.

The term “protected against infection” means that the immune system of a subject has been primed (e.g. by vaccination) to trigger an immune response and repel the infection. It will be clear to those skilled in the art that a vaccinated subject may thus get infected, but is better able to repel the infection than a control subject.

The term “treating” includes both therapeutic treatment and prophylactic or preventative treatment, wherein the object is to prevent or lessen infection. For example, treating may include directly affecting or curing, suppressing, inhibiting, preventing, reducing the severity of, delaying the onset of, reducing symptoms associated with, for example, infection, or a combination thereof. “Preventing” may refer, inter alia, to delaying the onset of symptoms, preventing relapse of a disease, and the like.

“Treating” may also include “suppressing” or “inhibiting” an infection or illness, for example reducing severity, number, incidence or latency of symptoms, ameliorating symptoms, reducing secondary symptoms, reducing secondary infections, prolonging patient survival, or combinations thereof.

Immunogenic compositions used as vaccines comprise an immunologically effective amount of antigen(s), as well as any other components, as needed. By “immunologically effective amount”, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Immunogenic compositions of the invention may include an antimicrobial, particularly when packaged in multiple dose formats.

Immunogenic compositions of the invention may include sodium salts (e.g. sodium chloride) to give tonicity. A concentration of 10±2 mg/ml NaCl is typical. In some embodiments, a concentration of 4 to 10 mg/ml NaCl may be used, e.g. 9.0, 7.0, 6.75 or 4.5 mg/ml.

### Methods of Treatment

The invention also provides a method of preventing or treating infection by Shigella in a subject, comprising administering an immunogenic composition of the invention to a subject. The invention also provides an immunogenic composition of the invention for use in prevention or treatment of infection by Shigella in a subject. The invention also provides the use of an immunogenic composition of the invention in the manufacture of a medicament for preventing or treating infection by Shigella in a subject.

The “preventing or treating infection by Shigella” in the method/immunogenic composition for use/use of the immunogenic composition in the manufacture of a medicament of the invention comprises raising an immune response in a subject. The immune response is preferably protective and preferably raises antibodies, more preferably IgG antibodies.

In an embodiment, the method/immunogenic composition for use/use of the immunogenic composition in the manufacture of a medicament of the invention will raise anti-S. sonnei antibodies and/or anti-S. flexneri antibodies. Preferably the antibodies will be IgG antibodies. In an embodiment, the method/immunogenic composition for use/use of the immunogenic composition in the manufacture of a medicament of the invention will raise anti-S. sonnei IgG antibodies and/or anti-S. flexneri IgG antibodies above a certain antibody titre threshold. In an embodiment, the method/immunogenic composition for use/use of the immunogenic composition in the manufacture of a medicament of the invention will increase the number of antibodies with anti-Shigella sonnei and/or Shigella flexneri bactericidal activity in a subject.

In an embodiment, the method/immunogenic composition for use/use of the immunogenic composition in the manufacture of a medicament of the invention wherein, on administration to a subject, the immunogenic composition elicits an increased number of antibodies with anti-Shigella sonnei and/or Shigella flexneri bactericidal activity compared to a composition that comprises a lower amount of O-antigen from corresponding Shigella sonnei and/or Shigella flexneri. In an embodiment, the immunogenic composition of the method/immunogenic composition for use/use of the immunogenic composition in the manufacture of a medicament of the invention comprises at least two times, three times, four times, five times, six times, seven times, eight times, nine times, or ten times as much O-antigen compared to the composition that comprises a lower amount of O-antigen from corresponding Shigella sonnei and/or Shigella flexneri. In an embodiment, the immunogenic composition of the method/immunogenic composition for use/use of the immunogenic composition in the manufacture of a medicament of the invention comprises up to and including two times, three times, four times, five times, six times, seven times, eight times, nine times, or ten times as much O-antigen compared to the composition that comprises a lower amount of O-antigen from corresponding Shigella sonnei and/or Shigella flexneri.

In an embodiment, the immunogenic composition of the method/immunogenic composition for use/use of the immunogenic composition in the manufacture of a medicament of the invention is suitably immunogenic such that, after immunisation with the immunogenic composition, a higher number of patients have at least a 4-fold increase in bactericidal activity compared with patients immunised with the composition that comprises a lower amount of O-antigen from corresponding Shigella sonnei and/or Shigella flexneri. In an embodiment, the immunogenic composition of the method/immunogenic composition for use/use of the immunogenic composition in the manufacture of a medicament of the invention is suitably immunogenic such that, after immunisation with the immunogenic composition, about or exactly twice, three times, four times or five times the number of patients have at least a 4-fold increase in bactericidal activity compared with patients immunised with the composition that comprises a lower amount of O-antigen from corresponding Shigella sonnei and/or Shigella flexneri.

The method/immunogenic composition for use/use of the immunogenic composition in the manufacture of a medicament of the invention may raise a booster response.

The immunogenic composition that is used to prevent or treat infection by Shigella is preferably able to raise an immune response in a subject and is more preferably a vaccine.

The method/immunogenic composition for use/use of the immunogenic composition in the manufacture of a medicament of the invention are preferably for the prevention and/or treatment of illness caused by Shigella e.g. shigellosis, dysentery and associated symptoms including diarrhoea, fever, abdominal pain, tenesmus, etc. These uses and methods are preferably for the prevention and/or treatment of illness caused by both Shigella sonnei and Shigella flexneri, optionally Shigella flexneri 1b, 2a and or 3a.

### Subject

The subject of the invention is a mammal, preferably a human. Where the vaccine is for prophylactic use, the human may be an adult i.e. subject is 18 years old or above 18 years old. Where the vaccine is for prophylactic use, the human may be a child i.e. below 18 years old. Where the vaccine is for prophylactic use, the child may be between 12 to 72 months, preferably between 24 to 59 months, more preferably between 6 to 12 months. Where the vaccine is for prophylactic use, the child is preferably around 9 months.

Where the vaccine is for therapeutic use, the human is preferably a child.

A vaccine intended for children may also be administered to adults e.g. to assess safety, dosage, or immunogenicity.

### Dosage Regimen

Immunogenic compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml. For human administration, the dose may be about 100 μg measured by protein, for example, delivered in a 0.5 ml dose at a concentration of 200 μg protein/ml.

The immunogenic compositions of the invention may be used to elicit systemic and/or mucosal immunity.

The immunogenic compositions of the invention may be administered in single or multiple doses. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

A single dose of the immunogenic compositions of the invention may be effective. Alternatively, one unit dose followed by a second unit dose may be effective. Typically, the second (or third, fourth, fifth etc.) unit dose is identical to the first unit dose. The second unit dose may be administered at any suitable time after the first unit dose, in particular after 1, 2 or 3 months.

When administering multiple doses, the disclosure herein refers to days on which the doses are given. A first unit dose would be administered on day 1. If a second unit dose is administered on “Day 100”, it refers to doing so 99 days after administering the first unit dose. If a subsequent third unit dose is given, for example on “Day 150”, it refers to doing so 149 days after the first unit dose, or 50 days after the second unit dose.

In an embodiment, a first unit dose of the immunogenic composition of the invention may be administered followed by a second unit dose. The first unit dose is administered on day 1. The second unit dose may be administered on any day between days 60 to 100, or days 120 to 250. The second unit dose may be administered around day 85, or around day 169. Both unit doses may be identical.

In an embodiment, a first unit dose of the immunogenic composition of the invention may be administered followed by a second unit dose, followed by a third unit dose. The first unit dose is administered on day 1. The second unit dose may be administered on any day between days 60 to 100. The second unit dose may be administered around day 85. The third unit dose may be administered on any day between days 200 and 300. The third unit dose may be administered around day 253. All unit doses may be identical.

Co-Administration with a Second, Different, Immunogenic Composition

The immunogenic composition of the invention may be co-administered with a second, different, immunogenic composition. The second, different, immunogenic composition is any composition that can raise an immune response in a subject, and prevent of treat an individual against an infection, but is an immunogenic composition that does not prevent or treat Shigella infection.

The second, different, immunogenic composition may comprise live, attenuated, measles virus and/or live, attenuated, rubella virus. The live, attenuated, measles virus may be derived from the Edmonston strain and the live, attenuated, rubella virus may be derived from the Wistar RA 27/3 strain.

Multiple doses of the second, different, immunogenic composition may be administered, preferably on separate days.

When a first unit dose of the immunogenic composition of the invention is administered on day one, and the second, different, immunogenic composition comprises live, attenuated, measles virus and/or live, attenuated, rubella virus, the first unit dose of the second, different, immunogenic composition may be administered between day 10 and 50, preferably around day 29. The second unit dose of the second, different, immunogenic composition may be administered between day 250 and 350, and preferably around day 281.

### EXAMPLES

### Example 1. Data from a Human Challenge Study Demonstrating that a Shigella sonnei Vaccine with a Low Dose of O-Antigen is not Sufficiently Immunogenic

A phase 2b study was conducted between August 2018 and November 2019, which evaluated safety, immunogenicity and efficacy of a Shigella sonnei GMMA candidate vaccine (1790GAHB) in adults, using a S. sonnei 53G controlled human infection model. Participants (randomized 1:1) received two doses of 1790GAHB or placebo (GAHB-Placebo), at day (D) 1 and D29, and an oral challenge of S. sonnei 53G at D57.

### Methods

Creating the NVGH1790 S. sonnei Strain

S. sonnei 53G [1] was chosen as parent strain. S. sonnei strain NVGH1859 (S. sonnei 53G ΔtolR::kan ΔvirG::nadAB) was obtained by replacing the plasmid-encoded virG gene [2] in S. sonnei 53G ΔtolR::kan [3] with the genes nadA and nadB from E. coli [18]. The upstream and downstream regions of virG were amplified using the primer pairs virGup-5/virGup-3 (upstream) and virGdown-5/virGdown-3 (downstream) (Table 1). The “nadAB” cassette was generated by amplifying nadA and nadB from E. coli using primers nadA-5/nadA-3 and nadB-5/nadB-3 (Table 1). The fragments were inserted into pBluescript (Stratagene) so that nadA and nadB were linked and interposed the flanking regions of virG. The replacement construct (virGup-nadAB-virGdown) was amplified using the primers virGup-5/virGdown-3 and used to transform recombination prone S. sonnei ΔtolR::kan as previously described [3].

S. sonnei strain NVGH1790 (S. sonnei 53G ΔtolR::kan ΔvirG::nadAB ΔhtrB::cat) was generated from NVGH1859 by replacing the htrB gene [4] by the chloramphenicol resistance gene cat as described by Rossi et al [5].

TABLE 1
Primers used for generation of S. sonnei GMMA producing strains

| | SEQ | |
|---|---|---|
| | ID | |
| Primer | No: | Sequence 5′-→3′ |
| virGup-5 | 1 | ACTCGAGCTCTGTAGTTGATTTGACAGTTGACATCC |
| virGup-3 | 2 | CTAACCCGGGCACTATATTATCAGTAAGTGGTTGATAAACC |
| virGdown- | 3 | CTAACCCGGGCGTGTTGATGTCCTGC |
| 5 | | |
| virGdown- | 4 | ACGCGTCGACAGTTCAGTTCAGGCTGTACGC |
| 3 | | |
| nadA-5* | 5 | CTAACCCGGGCAAGCAACTCTATGTCGGTGGAAT |
| nadA-3* | 6 | TATCAAGCTTGGCAAGGCCAATACACAGC |
| nadB-5* | 7 | TATCAAGCTTAGGGTTAGAGTGTCTCGTTTTTGTA |
| nadB-3* | 8 | CTAACCCGGGCCAGACCAGAACTATTCC |

*nadA and nadB primers as described by Prunier et al. [18] with small modifications

Production of GMMA from NVGH1790 S. sonnei Strain

### Fermentation

For each production batch, the Shigella strain was grown in a shake flask from the Research or GMP cell bank in SSDM at 30° C. with agitation (200 rpm), starting from an optical density measured at 600 nm (OD600) of 0.02 until the culture reached an OD600 equal to 1.5±0.5, usually in 9±2 hours. In the Bioreactor (30 L scale in Sartorius, Biostat D75 Bioreactor, or 25 L scale in LP35 Bioengineering Bioreactor), strains are cultured in Batch mode starting from 2% inoculum size with controlled cultivation conditions: pH 6.7 kept by addition of 28% NH₄OH, 30° C., dissolved oxygen kept at 30% saturation by 1 air volume per culture volume per minute (vvm) airflow, agitation and pressure in cascade (200-800 rpm, 50-1250 mbarg) until the final OD600 of 35.

### Purification

GMMA released into the fermentation broth were purified using two consecutive Tangential Flow Filtration (TFF) steps: a microfiltration in which the culture supernatant containing the GMMA is separated from the bacteria, and an ultrafiltration, in which the GMMA were separated from soluble proteins. For the microfiltration step (1.2 m² of a 0.2 μm pore size cellulose membrane), the bioreactor was connected with the TFF system, in order to use the fermentation vessel as a recirculation tank. The culture supernatant was initially concentrated three times to reach “one volume” of concentrated supernatant, followed by a discontinuous diafiltration against five volumes of the buffer in the growth medium (13.3 g/kg of KH₂PO₄; 4 g/kg of NH₄HPO₄; 1.7 g/kg of Citric acid; 4 mL/L of NH₄OH; pH 6.7). Physiological saline can also be used. The microfiltered material, containing GMMA, was then filtered through a filter capsule with 0.45 μm then 0.2 μm filters (Sartorius) to ensure absence of any viable Shigella bacteria before further processing. The ultrafiltration step (1.4 m² of a 300 kDa pore size PES membrane) consisted of concentration followed by continuous diafiltration of the microfiltered GMMA solution against ten volumes of Tris-buffered saline (TBS), 0.9% NaCl, 10 mM Tris/Tris HCl pH 7.4 or 0.9% w/v of sodium chloride, and permitted substantial removal of nucleic acids and soluble proteins. A final concentration of the purified GMMA was performed to obtain the required concentration for the formulation process and filtered through a Sartorius cellulose acetate sterilizing filter which was validated for extractables, leachables and bacteria retention capability with GMMA bulk. Three non-GMP consistency batches of S. sonnei GMMA were prepared from a fermentation volume of 30 L. Additionally, two GMP lots were produced and released to support the further manufacturing of toxicology and clinical vaccines. The bulk S. sonnei GMMA were tested for appearance, identity, total and soluble protein content, O antigen content, LPS content, pH osmolality, purity and size.

### Formulation

GMMA were adsorbed to aluminium hydroxide (ALHYDROGEL® 2%, Brenntag Biosector, Denmark) by adding the GMMA suspension to ALHYDROGEL® under constant stirring at room temperature for 2 h followed by vialing. The GMMA ALHYDROGEL® formulation contains 12.7 μg/mL S. sonnei O antigen, 200 μg/mL GMMA protein and 0.7 mg/mL aluminium-III-ion (Al³⁺) as ALHYDROGEL® in TBS. A histidine buffer can also be used. The formulation was dispensed at 0.7 mL per 3 mL single dose vial. The formulation was tested for identity, total protein content, aluminium content, extractable volume, non-adsorbed protein, visual appearance, pH, osmolality, sterility, immunogenicity, and pyrogenicity.

The resultant formulated GMMA vaccine (containing GMMA produced, purified and formulated as set out in the preceding paragraphs) is referred to as 1790GAHB. Three GMP lots of S. sonnei 1790GAHB, a toxicology lot and two clinical lots, were prepared and released. A smaller (140 mL) non-GMP stability lot was also generated. Freshly formulated small scale laboratory batches were produced for initial pyrogenicity and immunogenicity studies.

### GMP Formulation of GAHB-Placebo

Placebo, also used as diluent, was prepared containing 0.7 mg/mL Al³⁺ as ALHYDROGEL® in TBS and was dispensed at 0.7 mL per 3 mL vial. A histidine buffer can also be used. The GAHB-Placebo was tested for identity, aluminium content, extractable volume, visual appearance, pH, osmolality, sterility and pyrogenicity. Two GMP lots of GAHB-Placebo have been produced and released.

### Study Design

Participants were randomized (1:1) to receive 2 doses of either 1790GAHB (the study vaccine) (1790GAHB group) or placebo (Placebo group), 28 days apart, and were enrolled in four overlapping cohorts (Cohorts 1-4) for logistical reasons at study site, maintaining the 1:1 ratio. Both 1790GAHB and placebo were administered via intramuscular route, in the upper deltoid of the non-dominant arm. The study vaccine was provided as a preservative-free formulation (single vial of 0.7 mL) containing S. sonnei 1790-GMMA (12 μg/mL measured by OAg and 200 μg/mL measured by protein content) adsorbed to Alhydrogel (0.7 mg Al³⁺/mL) in Tris-buffered saline. The 0.5 mL dose containing 1·5/25 μg of OAg/protein was obtained by dilution with the placebo suspension (Alhydrogel in Tris-buffered saline [0.7 mg Al³⁺/mL]), immediately prior to vaccination.

A challenge dose of S. sonnei was administered at Day 57, 28 days post-second vaccination. The target challenge consisted of 1500 colony forming units (CFU) of reconstituted lyophilized S. sonnei strain 53G and was administered orally, after dilution of 1 mL of the inoculum in 30 mL of sterile saline solution (0.9%). The day before the challenge administration, participants were admitted to an inpatient unit where they had to stay for an 8-day period and were monitored daily. Eligibility was assessed before challenge and participants who did not meet the eligibility criteria or had taken antibiotics in the previous week were not administered the challenge agent and were discharged from the inpatient unit. Participants fasted for 90 minutes prior to receiving the challenge inoculum, and for an additional 90 minutes after challenge. To neutralize gastric acidity, participants drank a 120 mL solution of sodium bicarbonate approximately 5 minutes before drinking the challenge suspension. On the 5th day after challenge (or earlier in case of symptoms defined in appendix pp 1), participants received 500 mg of ciprofloxacin twice daily for 3 days. Participants received 500 mg of ciprofloxacin twice daily for 3 days earlier than on the 5th day after challenge if:
(i) they had diarrhoea (any severity stool Grade 3 or higher) and two or more of the following symptoms: severe abdominal pain/cramps, severe nausea, severe headache, severe myalgia, severe arthralgia, gross blood in ≥2 stools, any fever (≥38.0° C. measured orally), or any vomiting; and/or
(ii) they experienced unexpectedly severe events such as hypotension (disproportionate to volume loss), renal dysfunction, or altered mental state (e.g. somnolence) at the discretion of the investigators; and/or
(iii) a study physician determined early treatment is warranted for other reasons.

Blood samples were collected at screening, pre- and 7 days post-administration of each 1790GAHB/placebo dose, as well as pre- and 7 days, 28 days, and 6 months post-administration of the challenge inoculum. Stool samples were collected daily during the inpatient stay to verify presence of S. sonnei by culture and quantitative Polymerase Chain Reaction (qPCR) analysis. All stool samples were visually assessed for consistency and blood presence. Samples that were loose or watery (Grade 3-5) were weighed and if there was visible blood, a hemoccult test was performed to confirm presence of blood.

Randomization was performed using an internet randomization system, with a minimization procedure [6] to ensure a 1:1 balance between treatments. The study was observer-blinded and laboratory staff in charge of testing human samples were also blinded to the treatment, participant, and visit number.

The study was conducted in accordance with all applicable regulatory requirements, International Conference on Harmonisation-Good Clinical Practice guidelines, and the Declaration of Helsinki. The protocol and study-related documents were reviewed and approved by the CCHMC institutional review board on 10 May 2018. All participants provided electronic consent through REDcap system (www.projectredcap.org), except for one who provided written consent because the REDcap system was not working.

### Sample Size

Based on the percentage of participants with a seroresponse (defined as an increase in post-vaccination anti-S. sonnei LPS serum IgG concentrations of at least 50% for participants with pre-vaccination levels>50 enzyme linked immunosorbent assay (ELISA) units (EU), or an increase of at least 25 EU for participants with pre-vaccination levels≤50 EU) observed in previous studies, 14, 15 a VE of 70% was assumed. Based on the results obtained by CCHMC in volunteers challenged with 1500 CFU, an attack rate (AR) for the primary case definition of 58% in the placebo group was assumed. A total number of 21 confirmed cases was needed to demonstrate that the LL (lower limit) of the two-sided 90% CI for the VE was above 0% with 80% power (by one proportion power analysis, one-sided test, one-sided alpha=5%). Considering an AR of 58% in the placebo group and a percentage of non-evaluable participants of 22%, a sample size of approximately 72 individuals (36 per group/18 per cohort) was estimated to reach the 21 shigellosis cases. The sample size was determined using the PASS 12.0.2 software.

### Analysis of Vaccine Efficacy (VE)

All efficacy analyses were conducted in the per-protocol set (PPS), including all participants with available data in the full analysis set who correctly received the vaccine/placebo and had no major protocol deviation.

For the primary objective, VE was evaluated at the end of study period, as 1-risk ratio (RR) where RR is the proportion of participants meeting defined case definition for shigellosis in the 1790GAHB group over the proportion in the placebo group, together with 90% CIs. Additionally, Barnard's unconditional exact test was conducted (the LL of the 90% two-sided exact unconditional CI for VE calculated with the Miettinen-Nurminen method is above 0 if the p-value of the one-sided Barnard test is below 5%).

For secondary efficacy endpoints measured as proportion, vaccine efficacy and 90% confidence intervals (CIs) were calculated as outlined for the primary objective. For count endpoints (i.e. total number of Grade 3-5 stools), the risk ratio was estimated as the rate of Grade 3-5 stools in participants in the 1790GAHB group divided by the rate of Grade 3-5 stools in the Placebo group; 90% CI for this rate ratio were calculated from an unadjusted negative binomial model, to take into account possible over-dispersion of data, and the unadjusted p-value from the associated 2-tailed Wald was computed. Continuous endpoints (i.e. weight of Grade 3-5 stools per subjects, weight of Grade 3-5 stools per subject in those with at least a Grade 3-5 stools, and weight of Grade 3-5 stools accumulated from challenge to discharge per participant) were compared using unequal variance two-sample t-test in case of normality of data, unequal variance 2-sample t-test on the log values in cases where log-values were normally distributed, otherwise by Wilcoxon 2-sample test; unadjusted p-values from two-tailed test were computed. In cases of comparisons using log-values, the geometric mean as well as the arithmetic mean were calculated, with associated 90% CI. Participants with missing values of efficacy endpoints were excluded from analyses as they were considered missing completely at random, i.e. not informative and with no impact on inferences.

### Analysis of Safety

Solicited adverse events (AEs) and unsolicited AEs were assessed in the corresponding safety sets that included all vaccinated participants with available solicited/unsolicited safety data.

Participants were monitored for safety throughout the study. A GSK Safety Review Team analysed blinded data to detect potential safety signals on a continuous basis. At predefined timepoints, an Independent Data Monitoring Committee (IDMC) reviewed unblinded safety data and made recommendations concerning continuation, termination, or other modifications of the study. Based on prior experience in 1790GAHB-GMMA studies, 14,15 the occurrence of ≥ Grade 3 symptomatic neutropenia in ≥1 participant triggered a halting rule for enrolment of additional participants until the event was reviewed by IDMC. Grading was defined as absolute neutrophil count (ANC) 1800-1500 cells/μL (Grade 1), 1499-1000 cells/μL (Grade 2), 999-500 cells/μL (Grade 3), or <500 cells/μL (Grade 4).

Participants were observed for 30 minutes after each vaccination for occurrence of any immediate adverse event (AE). Solicited local (injection site pain, erythema, and induration) and systemic (arthralgia, chills, fatigue, malaise, myalgia, fever, headache) AEs were recorded on diary cards by study participants for 7 days post—each vaccination. Solicited AEs after the bacterial challenge were diarrhoea, abdominal pain, abdominal cramps, gas, anorexia, nausea, headache, myalgia, malaise, arthralgia, fever, fatigue, and vomiting, collected by the investigator up to 8 days post-challenge. Unsolicited AEs were collected up to 28 days post—each injection and 28 days post-challenge. All AEs were graded for severity on a scale from 0 to 3 or 4 by the investigator (FIG. 6). Serious AEs (SAEs) were collected during the entire study period. Causal relationship of unsolicited AEs or SAEs to 1790GAHB vaccine/placebo or challenge inoculum was also assessed by the investigator. All study participants with a neutropenia (ANC<1800 cells/μL) had additional blood draws on a weekly basis until resolution of neutropenia. Neutropenia was diagnosed by laboratory testing for complete blood count and was defined as a decrease of neutrophil count asymptomatically or symptomatically. Symptomatic neutropenia (according to investigator assessment) was considered as AE of special interest and collected during the entire study duration. The number and percentage of participants with AEs were calculated with their exact 95% CI.

### Analysis of Immunogenicity

Immunogenicity in terms of anti-S. sonnei LPS serum IgG geometric mean concentration (GMC) at each timepoint of blood sample collection and in terms of serum bactericidal activity (SBA) against S. sonnei prior to administration and 28 days post-administration of each 1790GAHB vaccine/placebo dose was evaluated.

Immunogenicity analyses were conducted in the per-protocol set. Results were presented overall and separately for participants who did or did not develop shigellosis (primary case definition) during the 8-day inpatient period. The antibody response cut-off of 268 ELISA units (EU)/mL used in this assay corresponds to the formerly used threshold of 121 EU [7] which was estimated to correspond to the median endpoint titre of 1:800 reported in the sera of convalescent individuals previously infected with S. sonnei [8].

Anti-S. sonnei LPS serum IgG was measured at D1, 7, and 28 days post—each 1790GAHB/placebo dose and post-challenge. For each group, geometric mean concentrations (GMCs) and their 95% CIs were computed by exponentiating (base 10) the mean and 95% CIs of the log 10 ELISA concentrations. Median anti-S. sonnei LPS IgG concentrations were also calculated. The number and percentage of participants with anti-S. sonnei LPS IgG≥268 EU/mL with related 95% CIs were also calculated. Bactericidal activity was assessed by a luminescent serum bactericidal assay (L-SBA) [9] at D1 and 28 days post—each vaccination. SBA geometric mean titres (GMTs) were tabulated with their 95% CIs. Antibody concentrations below the lower limit of quantification (LLOQ) (22 EU/mL for ELISA and inhibition concentration [IC]50 of 100 for L-SBA) were set to half that limit for the purpose of analysis. Within-subject geometric mean ratios were computed for GMC/GMT at each post-vaccination time points versus baseline levels and post-challenge versus pre-challenge by exponentiating the mean within-subject differences in log-transformed titres and the corresponding CIs. All statistical analyses were carried out using Statistical Analysis Systems 9.4.

### Immunogenicity Results

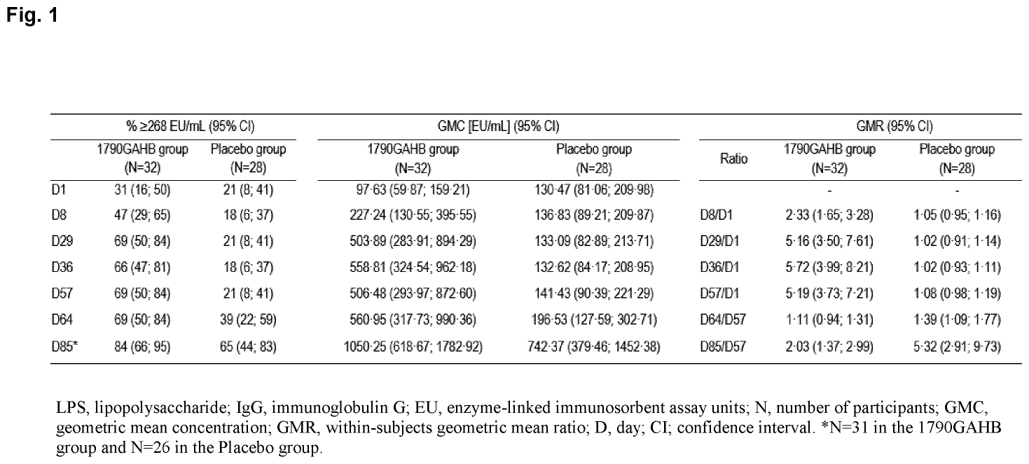

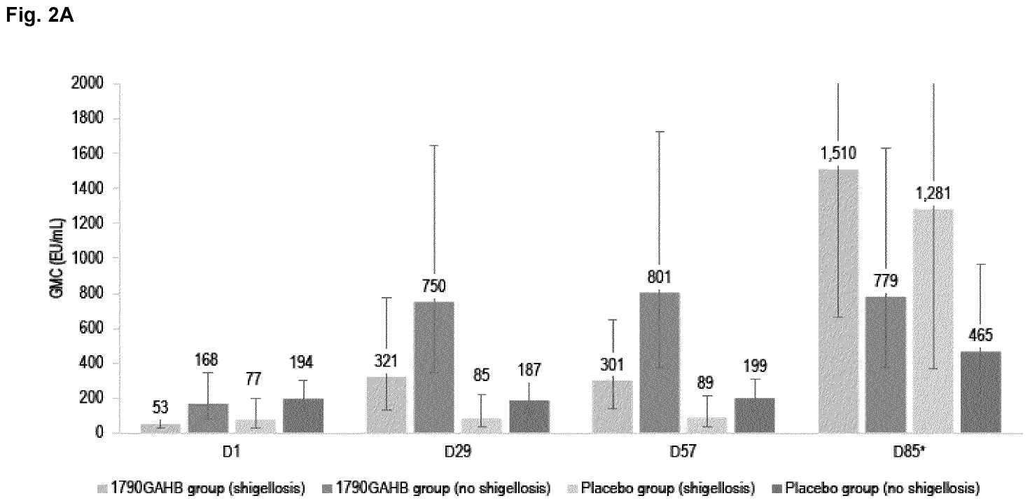

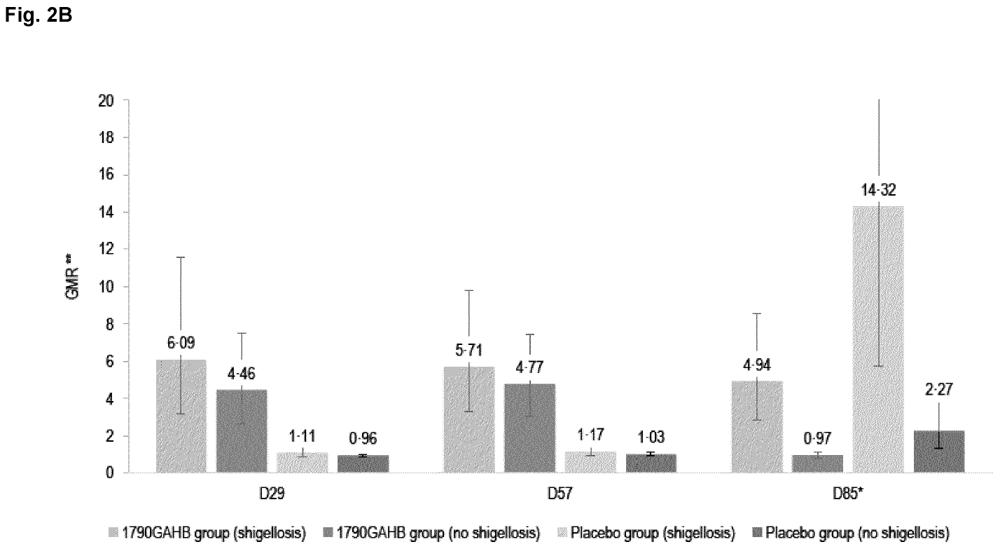

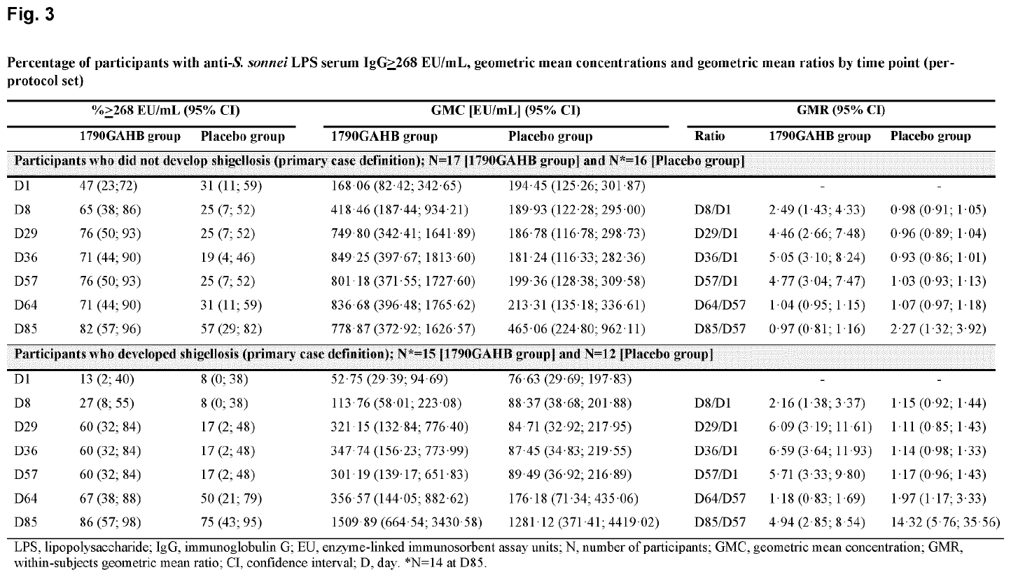

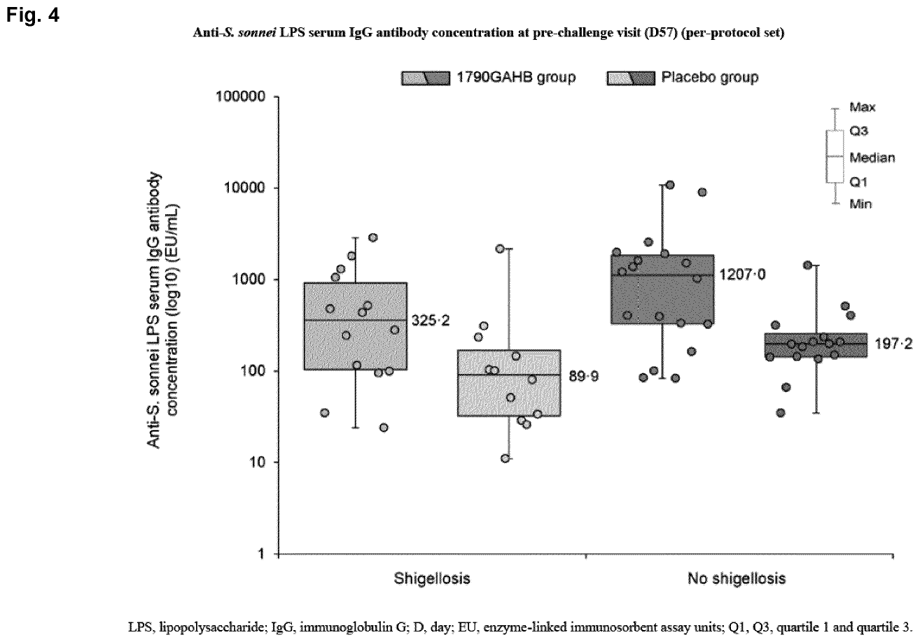

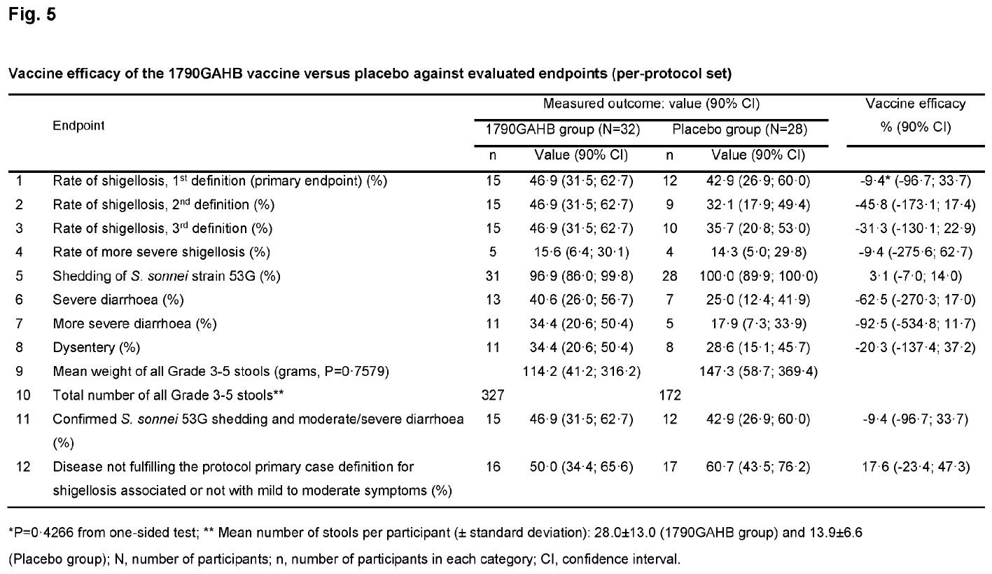

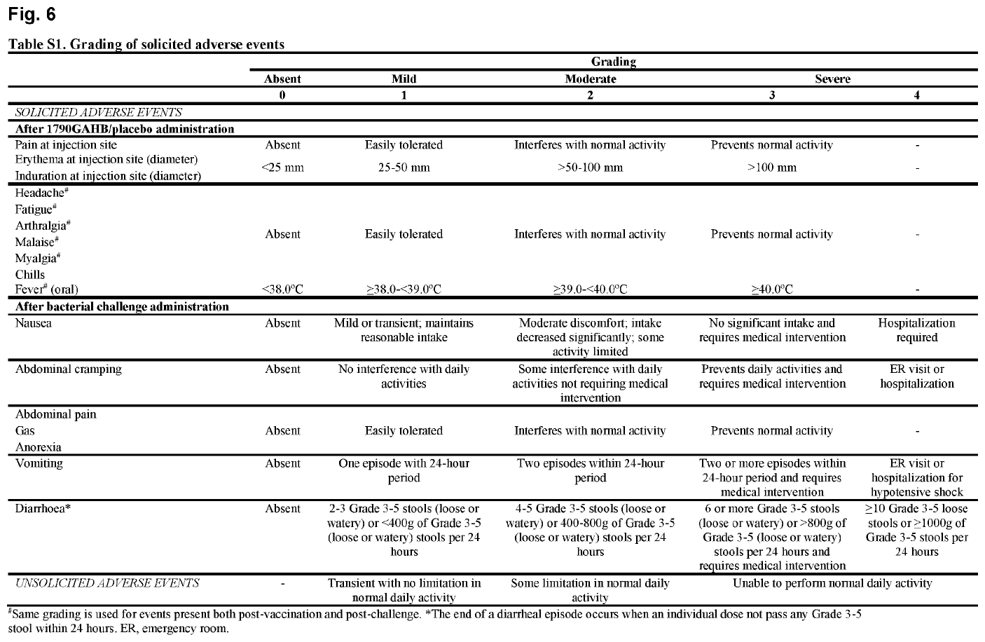

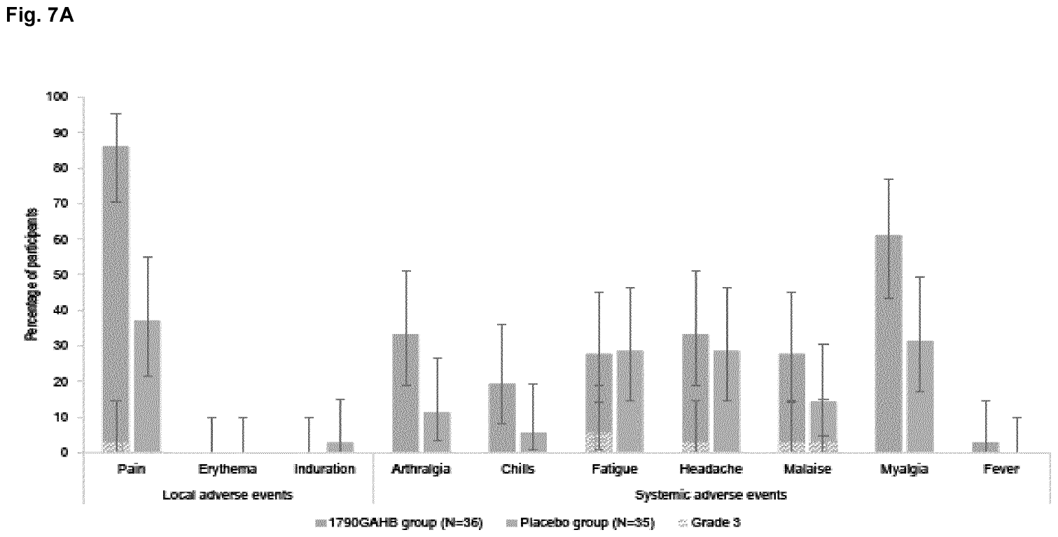

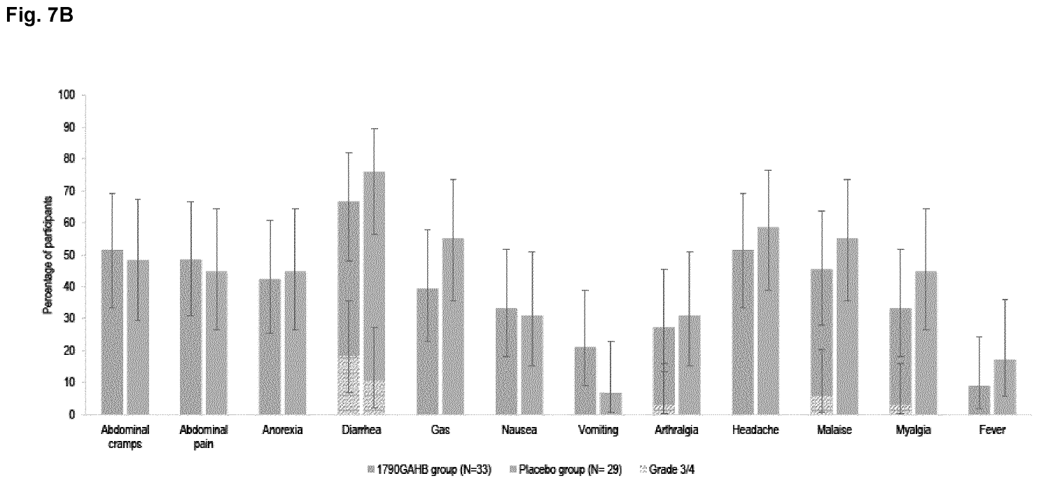

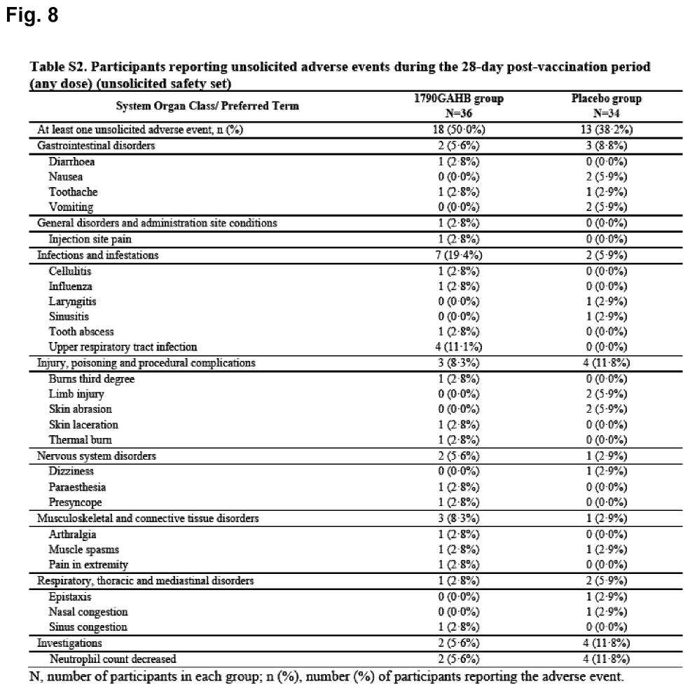

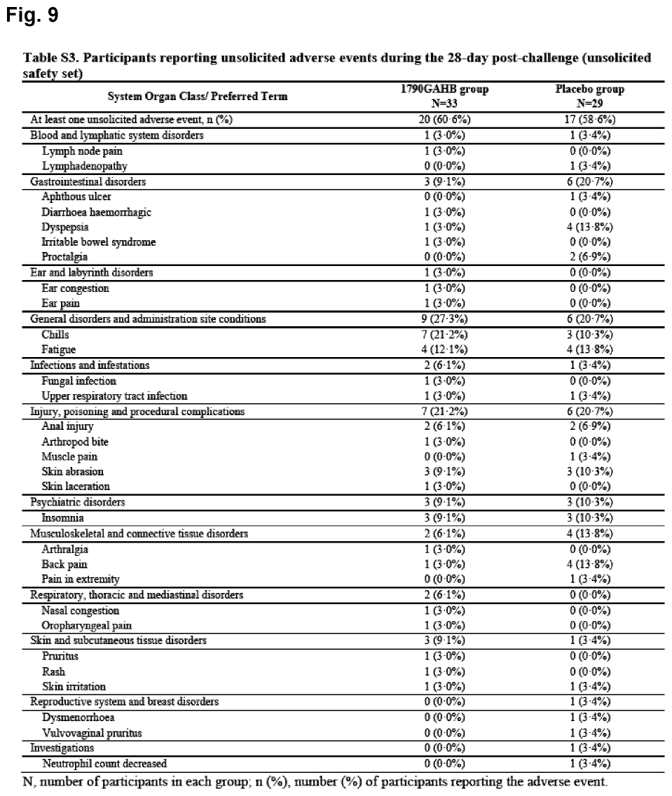

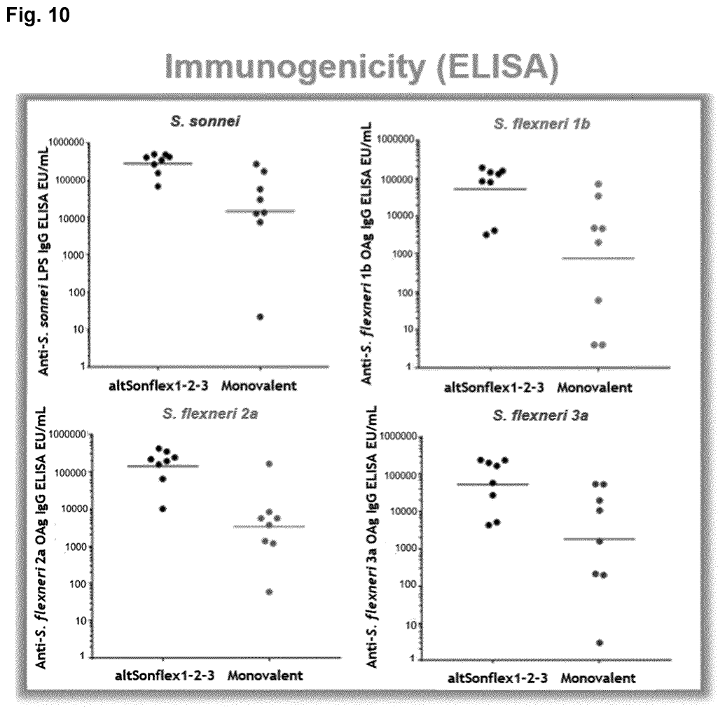

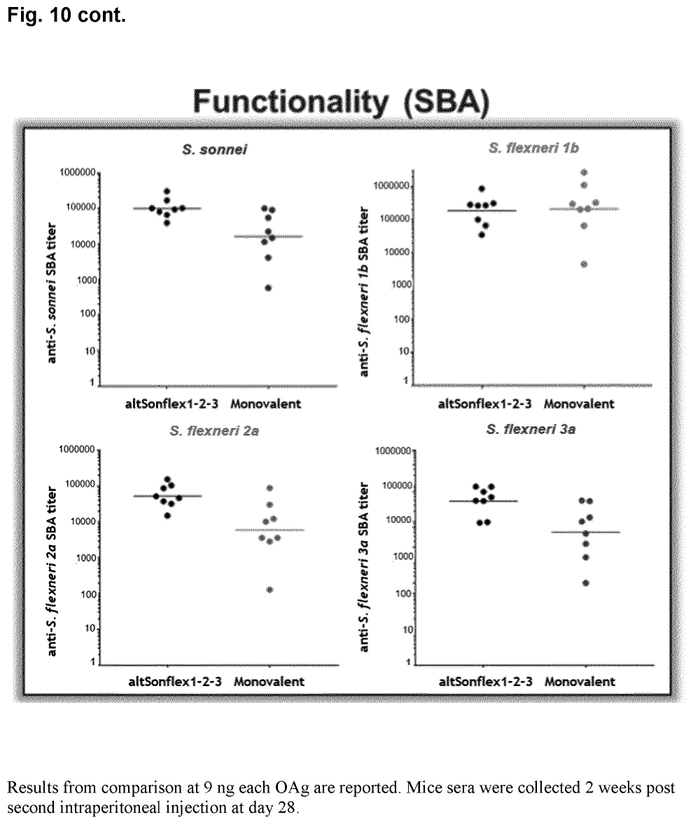

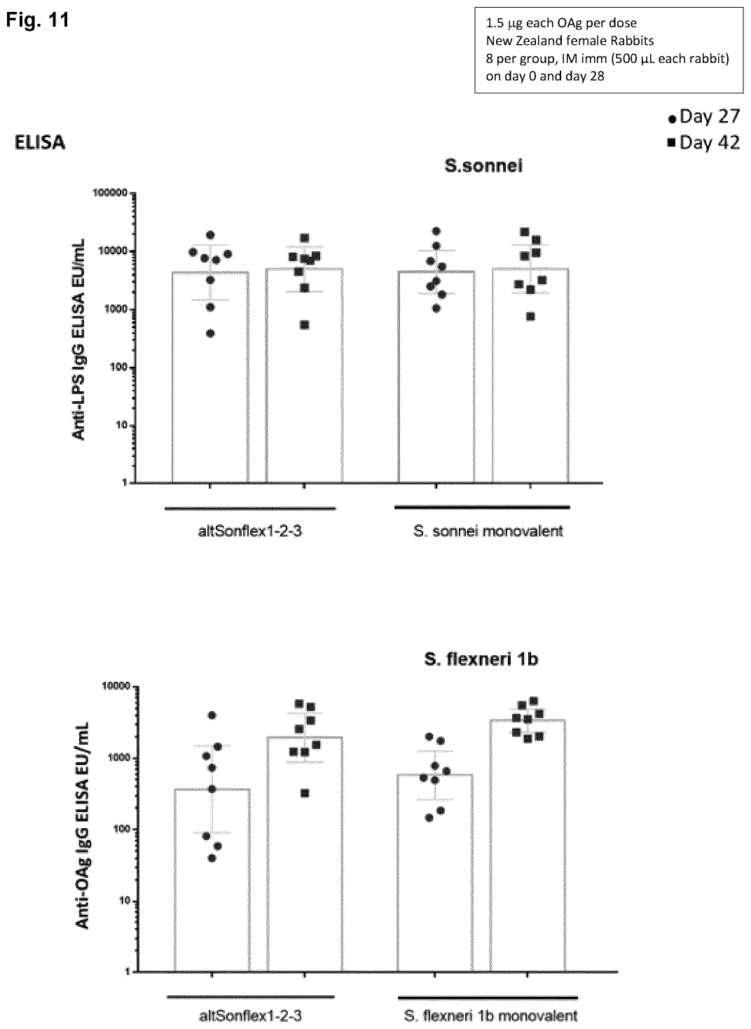

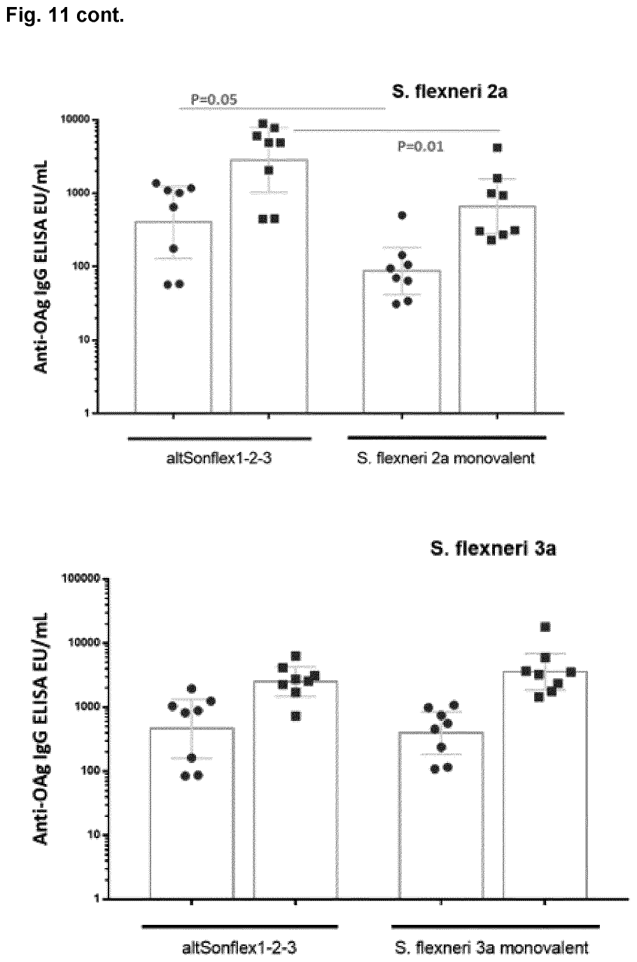

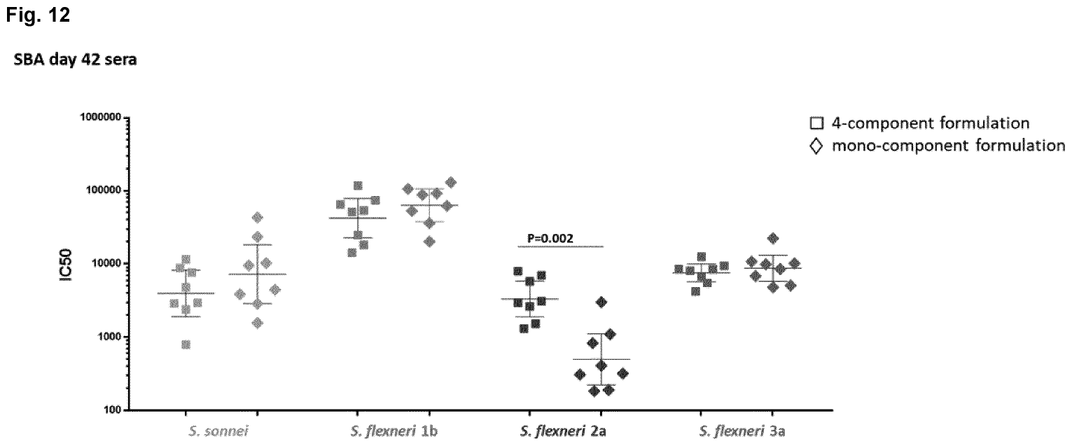

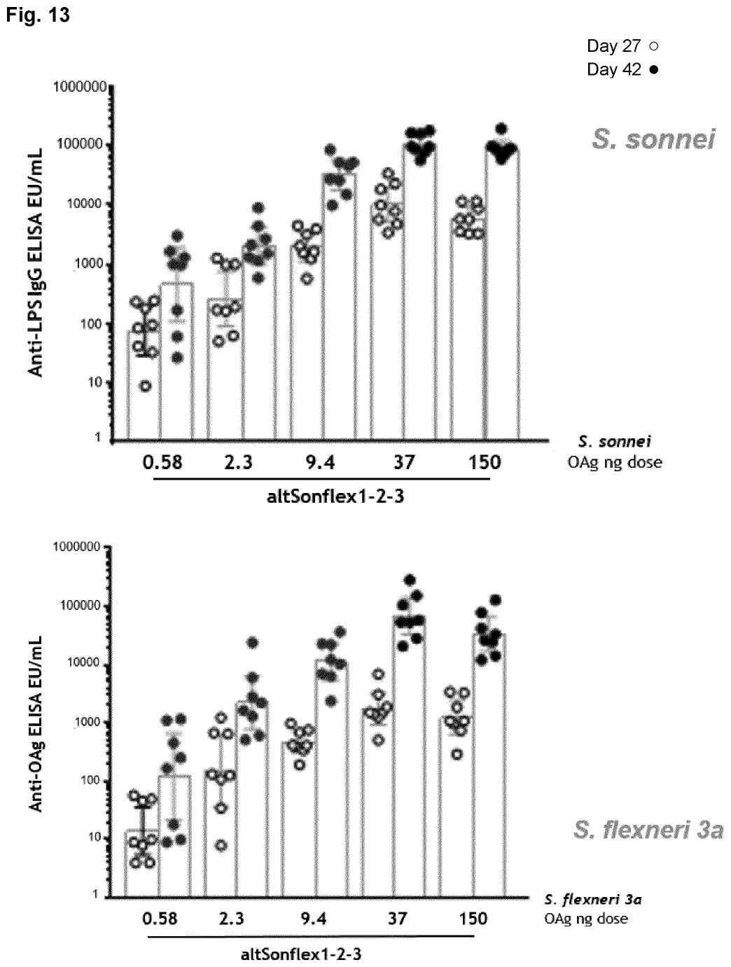

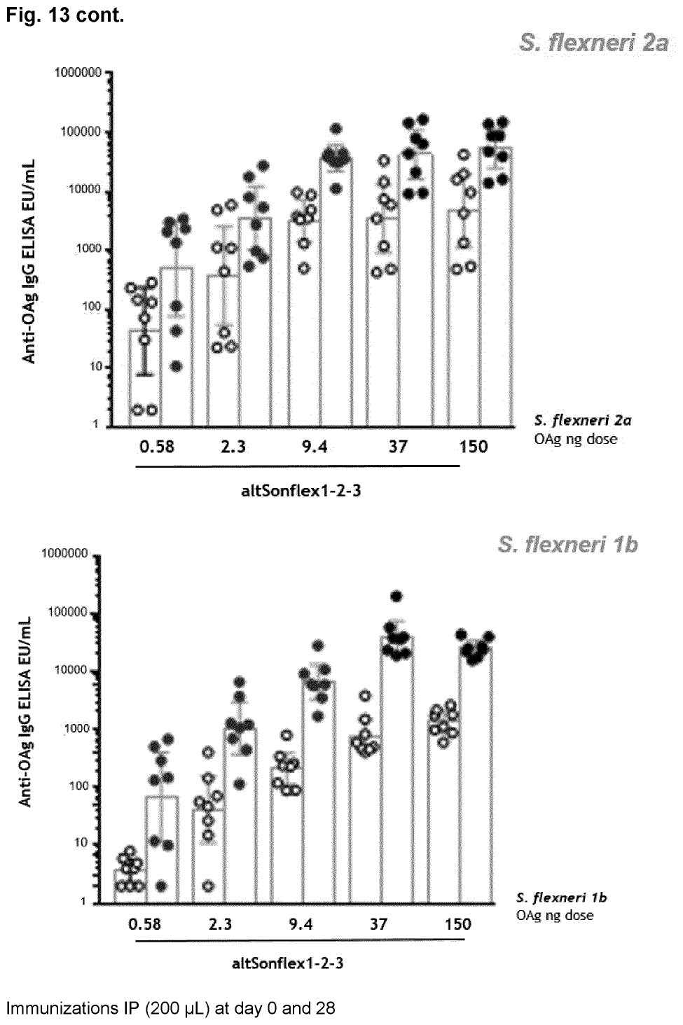

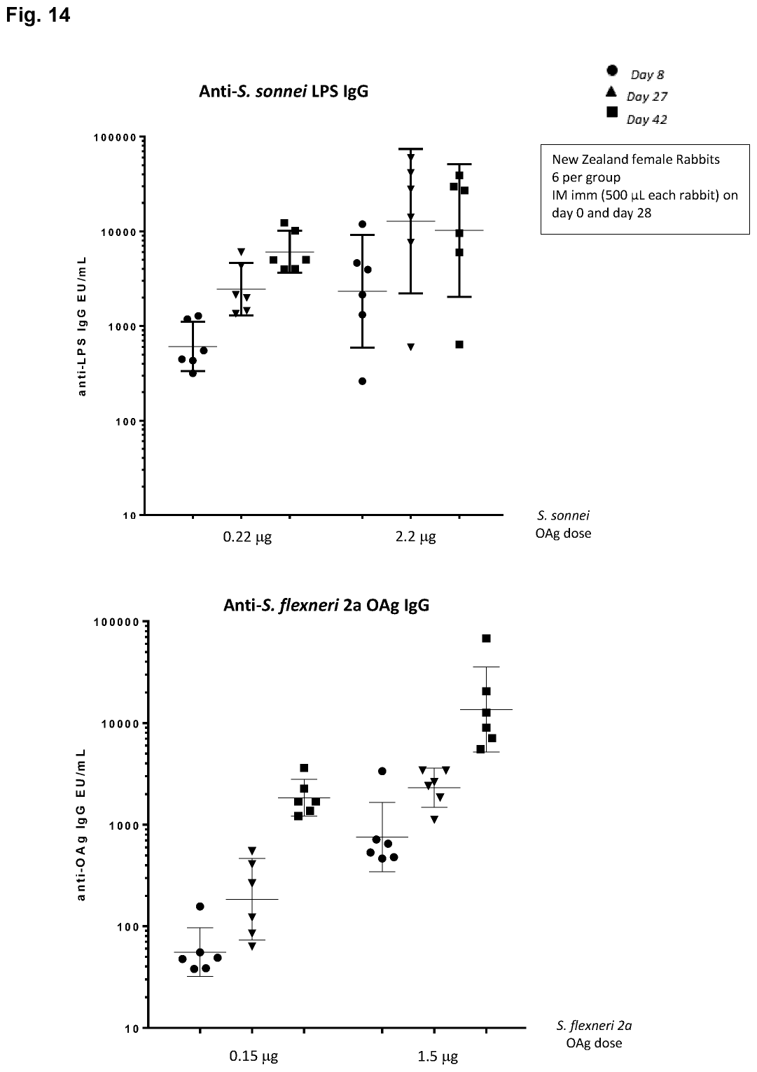

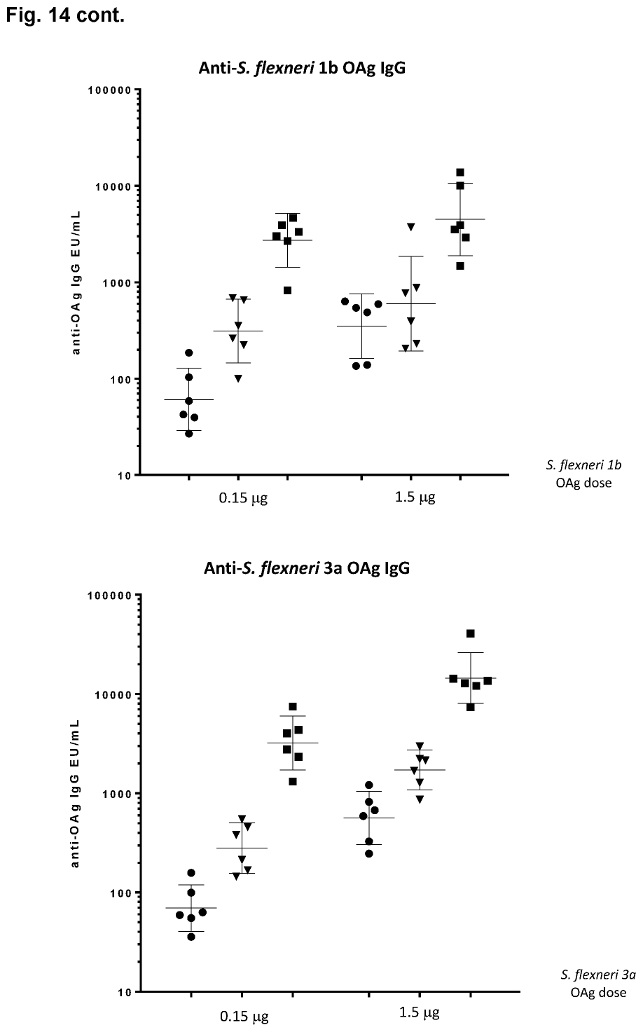

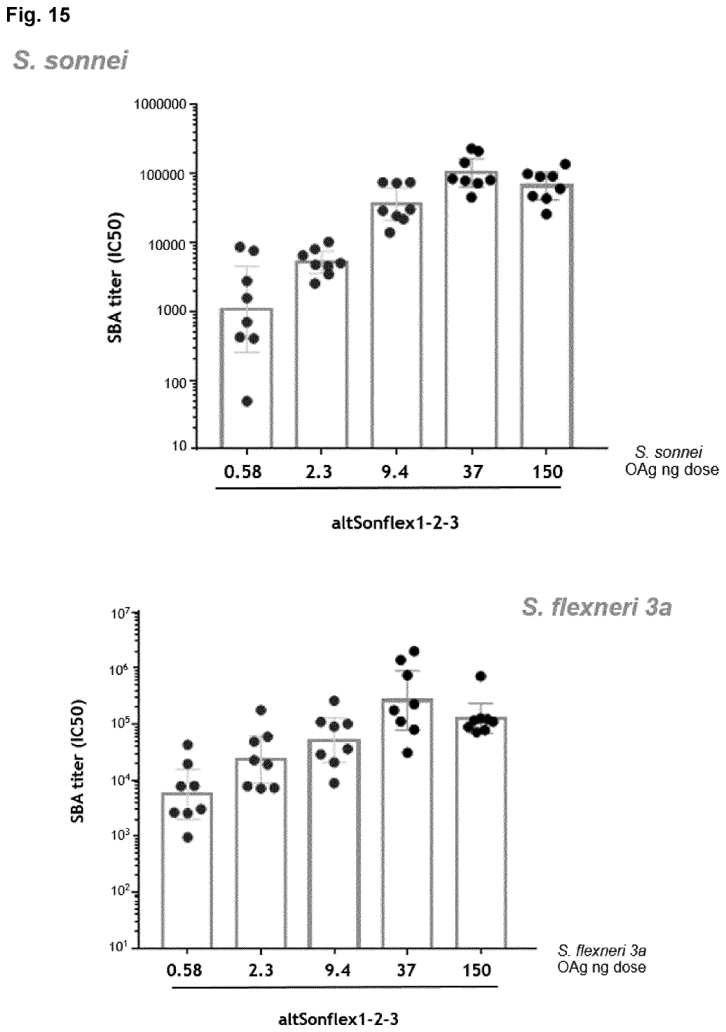

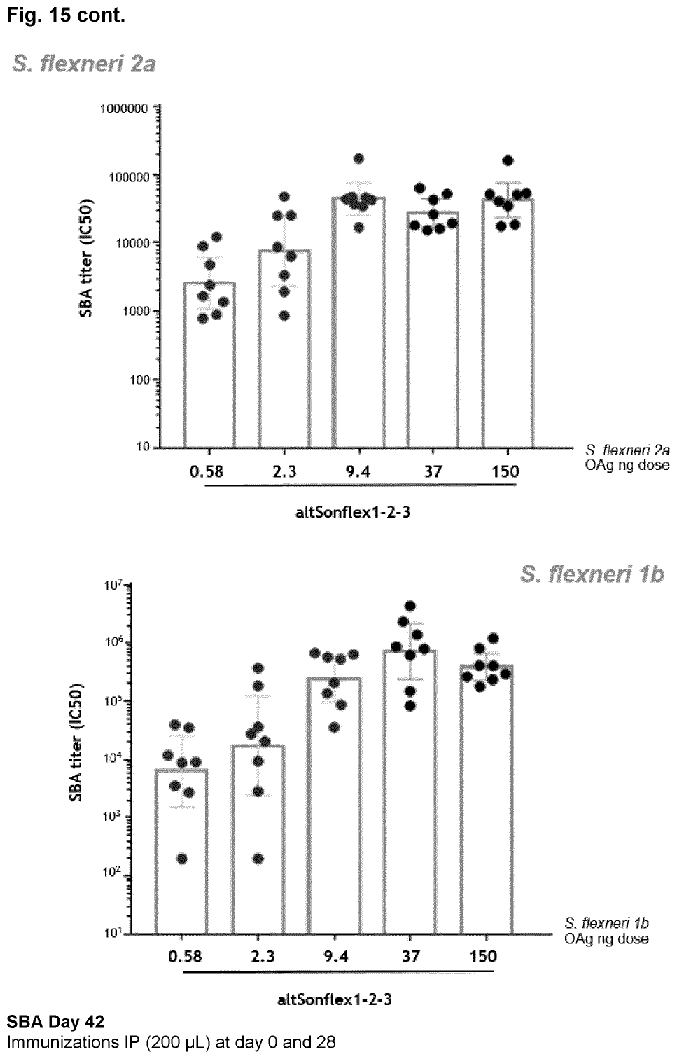

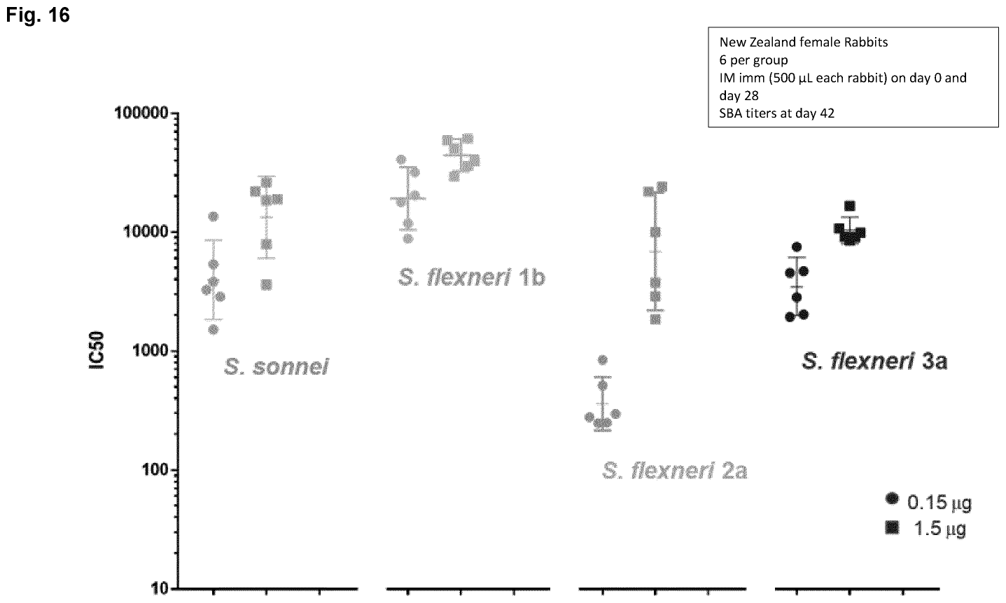

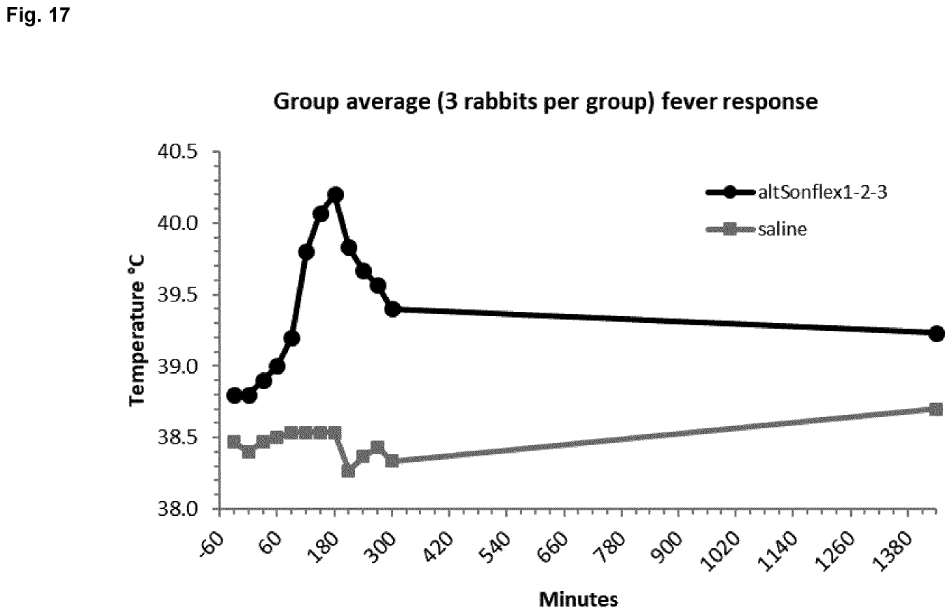

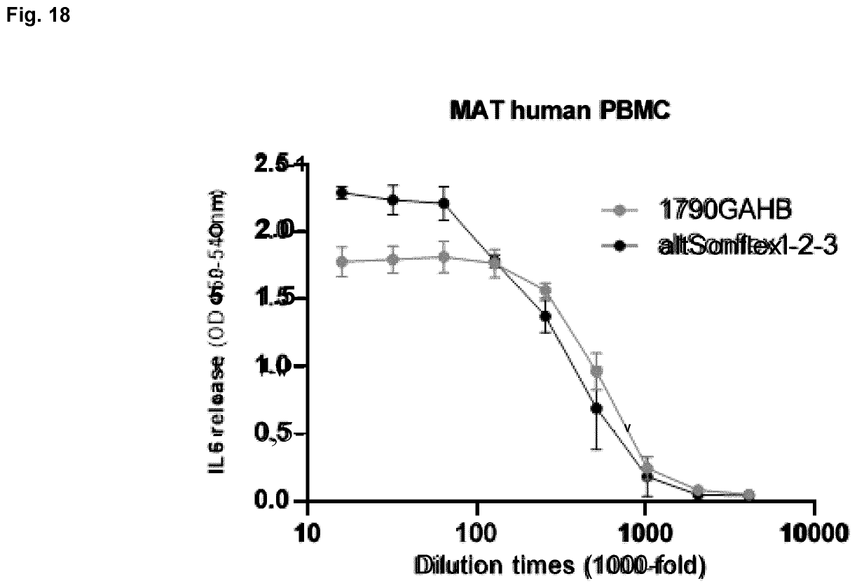

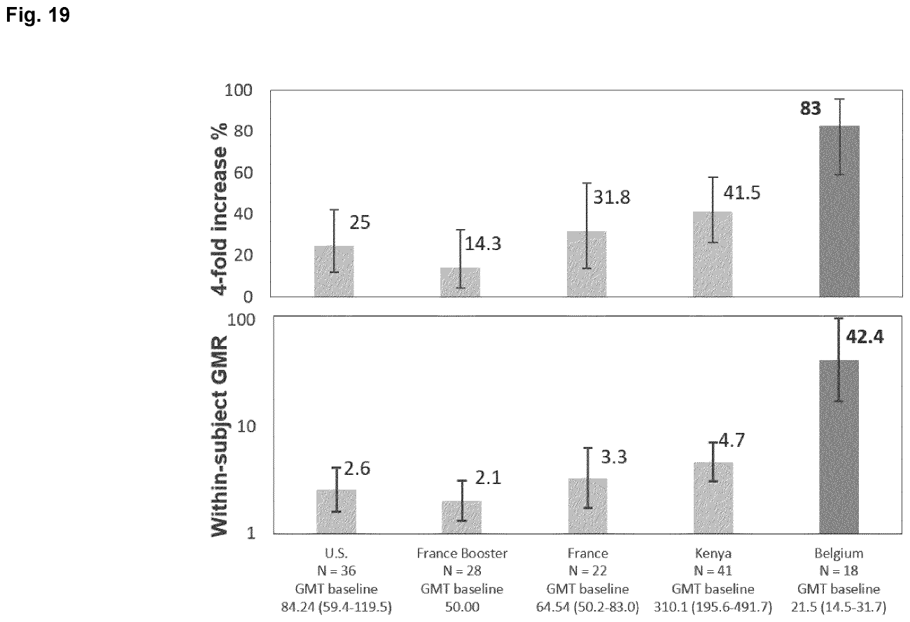

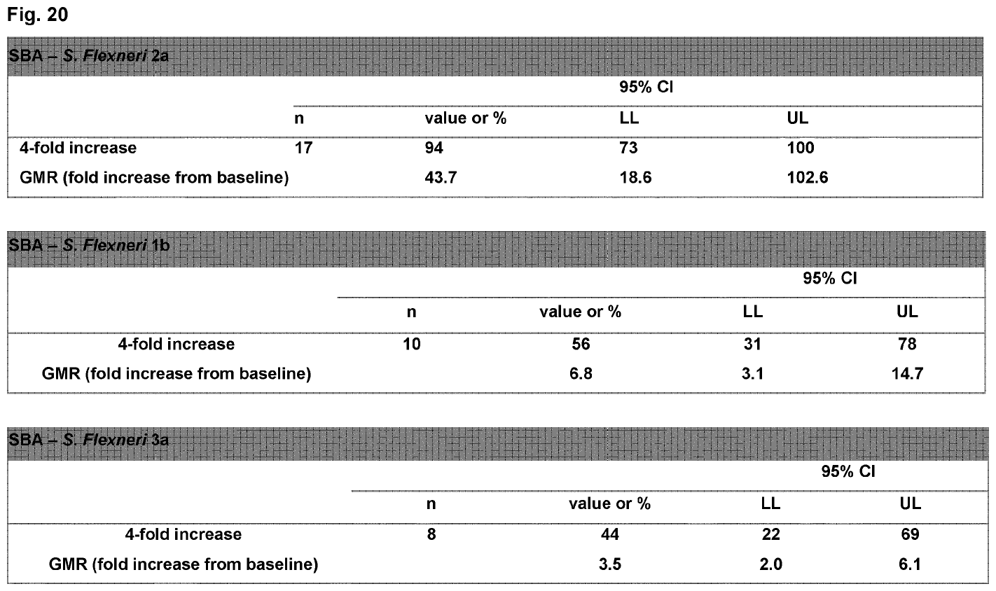

Baseline anti-S. sonnei LPS serum IgG GMCs were 97.6 EU/mL in the 1790GAHB group and 130.5 EU/mL in the Placebo group (FIG. 1). In both groups, baseline antibody levels were higher among participants who did not develop versus those who developed shigellosis post-challenge: 168·1 versus 52.8 EU/mL (P=0.0133 from t-test on log₁₀ transformed data) in 1790GAHB and 194.5 versus 76.6 EU/mL (P=0.0690) in placebo recipients (FIGS. 2 and 3). At 28 days post-first vaccination (D29), in the 1790GAHB group, GMC increased to 749.8 EU/mL in participants who did not develop shigellosis post-challenge and 321·2 EU/mL in participants who developed shigellosis post-challenge. No substantial increase in antibody levels was shown at D57, after the second vaccination. No increases in antibody GMCs were observed after any dose in the Placebo group, but, as for the 1790GAHB group, the pre-challenge median anti-S. sonnei LPS serum IgG concentration was higher among participants who did not develop shigellosis compared to participants who developed the disease following the challenge dose (P=0.1050 [Wilcoxon test] in the 1790GAHB group and P=0.0373 in the Placebo group) (FIG. 4).

### Vaccine Efficacy Results

During the 8-day post-challenge period, 15 participants in the 1790GAHB group and 12 in the Placebo group developed shigellosis according to the primary case definition, resulting in an AR of 46.9% (1790GAHB group) and 42.9% (Placebo group). VE was-9.4% (90% CI: −96.7-33.7; P=value 0.4266) (FIG. 5). As the LL of the 90% CI for VE is below 0, no efficacy was demonstrated according to the primary endpoint. Efficacy was not demonstrated for any other secondary endpoints, including lack of significant difference in quantitative shedding (measured by qPCR as CFU/g per day) between vaccinated and placebo groups (Wilcoxon rank test P=0.6858).

### Safety Results

During the 7-day post-vaccination period, 31 (86·1%) participants in the 1790GAHB group and 13 (37·1%) participants in the Placebo group reported at least one solicited local AE and 24 (66·7%) and 17 (48·6%) participants, respectively, reported at least one solicited systemic AE. All solicited local AEs were mild to moderate in severity (grading of AEs detailed in FIG. 6) except for one case of Grade 3 pain in the 1790GAHB group (FIG. 7A). The most frequent systemic AE was myalgia in both groups. All reported solicited systemic AEs were mild to moderate in severity except for four Grade 3 AEs in the 1790GAHB group after the second dose (fatigue [2 participants], headache [1 participant], and malaise [1 participant]) and one Grade 3 AE (malaise) in the Placebo group after the first dose. One Grade 1 fever event (≥38.0° C.-<39.0° C.) was observed in the 1790GAHB group after the first dose.

During the 8-day post-challenge period, the most frequently reported solicited event was diarrhoea in 22 (66·7%) participants in the 1790GAHB group and 22 (75·9%) in Placebo group. Grade 4 diarrhoea was reported by 6 (18·2%) vaccines and 3 (10·3%) placebo recipients (FIG. 7B).

During the 28-day post-vaccination period, 18 (50·0%) participants in the 1790GAHB group and 13 (38·2%) participants in the Placebo group reported at least one unsolicited AE (FIG. 8); 5 (13·9%) and 3 (8·8%) participants, respectively, experienced an event considered by the investigator as causally related to vaccine/placebo administration.

During the 28-day post-challenge period, at least one unsolicited AE was reported by 20 (60·6%) participants in the 1790GAHB group and 17 (58·6%) participants in the Placebo group (FIG. 9); 12 (36·4%) and 12 (41·4%) participants, respectively, experienced an event considered causally related to challenge dose. No severe unsolicited AE was reported during the 28-day post-vaccination/challenge periods. No deaths, nor AE of special interest (symptomatic neutropenia) were reported during the study. However, eight events of Grade 1 neutropenia (i.e., absolute neutrophil count [ANC] 1800-1500 cells/μL) were reported in 2 subjects in the 1790GAHB group; nine events of Grade 1 neutropenia and three events of Grade 2 neutropenia (i.e., ANC 1499-1000 cells/μL) were reported in 5 subjects in the Placebo group. All events were transient and clinically asymptomatic. The neutrophil values returned above the threshold values set in the study protocol, with a duration between 1 and 21 days after detection.

No serious AE (SAE) was reported in the 1790GAHB group. In the Placebo group, one participant experienced four SAEs following the challenge dose: deep vein thrombosis (moderate intensity) and pelvic venous thrombosis (severe) at day 107, hematoma (severe) at day 121, and carotid artery aneurysm at day 130 from challenge. All events were assessed by the investigator as unrelated to any study treatment and were resolved.

There were no AEs that led to subject premature withdrawal from the study, any dose reduction, interruption, or delay in study vaccination.

### Example 2. Formulation of the altSonflex 1-2-3 Vaccine

The following examples concern a multivalent Shigella GMMA vaccine, containing GMMA from S. sonnei, and S. flexneri 1b, 2a and 3a (also referred to as altSonflex 1-2-3 herein).

S. sonnei Strain Generation

S. sonnei 53G was chosen as the parent strain. S. sonnei strain NVGH1859 (S. sonnei 53G ΔtolR::kan ΔvirG::nadAB) was obtained first by replacing the tolR gene with the kanamycin resistance gene kan, as described by Berlanda Scorza et al [3]. Subsequently, the virulence plasmid-encoded virG gene was replaced with the nadA and nadB genes from E. coli, as described by Gerke et al [10].

S. sonnei strain NVGH2929 (S. sonnei 53G ΔtolR::kan ΔvirG::nadAB ΔmsbB2::cat ΔmsbB::erm) was generated from NVGH1859 by replacing the msbB1 and msbB2 genes with the erytromicin and chloramphenicol resistance genes erm and cat, as described by Mancini et al [11]. All primers used are listed in Table 2.

TABLE 2
Primers used in this study for generation of S. sonnei GMMA producing
strain

| | Deletion | SEQ ID | |
|---|---|---|---|
| Primer | construct | No: | Sequence 5′-3′ |
| tolR.Kan. | tolR::kan | 9 | TCTGGAATCGAACTCTCTCG |
| 500-5 | | | |
| tolR.Kan. | | 10 | ATTTTGAGACACAACGTGGCTTTCATGGCTTAC |
| L3 | | | CCCTTGTTG |
| tolR.Kan. | | 11 | TTCACGAGGCAGACCTCATAAACATCTGCGTTT |
| L5 | | | CCCTTG |
| tolR.Kan. | | 12 | TTGCTTCTGCTTTAACTCGG |
| 500-3 | | | |
| ampli.Kan- | | 13 | ATGAGCCATATTCAACGGGAAAC |
| 5 | | | |
| ampli.Kan- | | 14 | TTAGAAAAACTCATCGAGCATCAAA |
| 3 | | | |
| virGup-5 | virG::nadAB | 15 | ACTCGAGCTCTGTAGTTGATTTGACAGTTGACA |
| | | | TCC |
| virGup-3 | | 16 | CTAACCCGGGCACTATATTATCAGTAAGTGGTT |
| | | | GATAAACC |
| virGdown- | | 17 | CTAACCCGGGCGTGTTGATGTCCTGC |
| 5 | | | |
| virGdown- | | 18 | ACGCGTCGACAGTTCAGTTCAGGCTGTACGC |
| 3 | | | |
| nadA-5 | | 19 | CTAACCCGGGCAAGCAACTCTATGTCGGTGGA |
| | | | AT |
| nadA-3 | | 20 | TATCAAGCTTGGCAAGGCCAATACACAGC |
| nadB-5 | | 21 | TATCAAGCTTAGGGTTAGAGTGTCTCGTTTTTG |
| | | | TA |
| nadB-3 | | 22 | CTAACCCGGGCCAGACCAGAACTATTCC |
| XbaI.msbB. | msbB1::erm | 23 | CTAGTCTAGAAGTGCTTTCAGTGGGTGACG |
| 59.F | | | |
| EcoRV.msbB. | | 24 | AGCTTGATATCCCATGCTTTTCCAGTTTCGG |
| 59.R | | | |
| EcoRV.msbB. | | 25 | AGCTTGATATCGGCGAAATCCAACCGTATAAG |
| 39.F | | | |
| XhoI.msbB. | | 26 | CCGCTCGAGGGGGAAGTTGTTAAGACAGAC |
| 39.R | | | |
| EcoRV.Ery. | | 27 | AGCTTGATATCAGAGTGTGTTGATAGTGCAGTA |
| F | | | TC |
| EcoRV.Ery. | | 28 | AGCTTGATATCACCTCTTTAGCTTCTTGGAAGC |
| R | | | T |
| msbB2 | msbB2::cat | 29 | TAAAATATTAATGATGATTATGGTAGGGGCATT |
| KO F | | | CGCACTAAATAATGAAAGTGTAGGCTGGAGCT |
| | | | GCTTC |
| msbB2 | | 30 | ACAACTAGTGGAAATACCTGTACTTTATAATTT |
| KO R | | | CAAGGGTACGGGTCCGCCATATGAATATCCTC |
| | | | CTTAG |

S. flexneri 2a Strain Generation

S. flexneri 2a 2457T was chosen as parent strain. Before the generation of mutants, a white colony was selected on Congo red agar, indicating the loss of the virulence plasmid pINV. The curing of pINV was confirmed by the absence of the origin of replication (ori) or plasmid-encoded genes using PCR.

S. flexneri 2a strain NVGH2404 (S. flexneri 2457T ΔtolR::kan, ΔmsbB::cat) was obtained as previously described by Rossi et al [5]. Briefly, the tolR gene was replaced with the kanamycin resistance gene kan using the same strategy as primers described for S. sonnei. Subsequently, the msbB gene was replaced with the chloramphenicol resistance gene cat, using the primers listed in Table 3. Contrarily to S. sonnei strain NVGH2929, the msbB2 mutation was not needed as the gene is located on the cured virulence plasmid.

S. flexneri 3a and 1b Strains Generation

Shigella flexneri 3a 6885 and Shigella flexneri 1b Stansfield were chosen as parent strains. As for S. flexneri 2a, white colonies were selected on Congo red agar before the start of the genetic modification, indicating the loss of the virulence plasmid pINV. S. flexneri 3a strain NVGH2766 (S. flexneri 6885 ΔtolR::kan, ΔmsbB::cat) was generated using the same strategy and primers as described for strain NVGH2404. S. flexneri 1b strain NVGH2858 (S. flexneri Stansfield ΔtolR::frt ΔmsbB1a::frt ΔmsbB1b::frt) was prepared by adapting the methods described in Datsenko et al [12]. Briefly, the tolR gene and two chromosomal copies of the msbB genes were replaced with the kanamycin resistance gene kan, using the primers listed in Table 3. Removal of the antibiotic selective marker was performed after each gene deletion using the plasmid pCP20.

TABLE 3
Primers used in the generation of S. flexneri 1b, 2a and 3a mutants

| Primer | Deletion | SEQ ID | |
|---|---|---|---|
| name | construct | No: | Sequence 5′-3′ |
| msbB- | msbB::cat | 31 | CTAGTCTAGAAACCCGGGTGATAGTGTAGCGGCACA |
| flexU5 Sma | (S. flexneri | | |
| msbB- | 2a and 3a) | 32 | ACTCGAGCTCGTGAGCAAAGCCAGCTG |
| flexU3 Sac | | | |
| msbB- | | 33 | ACGCGTCGACCTCGGTGTGGAAATTGG |
| flexD5 SalI | | | |
| msbB- | | 34 | CTAACCCGGGCAACGTACTTACTCTACCG |
| flexD3 Xba | | | |
| Sma | | | |
| EcoRV.Cm | | 35 | AGCTTGATATCTGTGACGGAAGATCACTTCG |
| F | | | |
| EcoRV.Cm | | 36 | AGCTTGATATCGGGCACCAATAACTGCCTTA |
| R | | | |
| 47- | tolR::frt | 37 | ACCGCCAGGCGTTTACCGTTAGCGAGAGCAACAAG |
| tolRKOF | (S. | | GGGTAAGCCATGGCCGTGTAGGCTGGAGCTGCTTC |
| 48- | flexneri 1b) | 38 | ACCCGCTCTCTTTCAAGCAAGGGAAACGCAGATGTT |
| tolRKOR | | | TAGATAGGCTGCGTCATATGAATATCCTCCTTAG |
| 51- | msbB1a::frt | 39 | ACTATCACCAGATTGATTTTTGCCTTATCCGAAACTG |
| msbBKOF | (S. | | GAAAAGCATGGAAGTGTAGGCTGGAGCTGCTTC |
| 52- | flexneri 1b) | 40 | TTTTATTTGATGGGATAAAGATCTTTGCGCTTATACG |
| msbBKOR | | | GCTGGATTTCGCCCATATGAATATCCTCCTTAG |
| 0147- | msbB1b::frt | 11 | CGCCACCCGCGCTACTGGGGAGCATGGCTGGGTGTA |
| msbBKOF1 | (S. | | GCAGCGATGGCGGGGTGTAGGCTGGAGCTGCTTC |
| 0148- | flexneri 1b) | 42 | GCGAGTTTTCAGCAATTTTAGTATCCAGGTGTATTGT |
| msbBKOR1 | | | TCTGGTCGCGGACCATATGAATATCCTCCTTAG |

S. sonnei and S. flexneri 1b, 2a and 3a GMMA Production and Purification.

For each production batch, the Shigella strains are grown in a shake flask from a Research or GMP cell bank in defined medium (DM) at 30° C. with agitation (200 rpm), starting from an appropriate optical density measured at 600 nm (OD600) until the culture reaches an OD600 equal to 4.5±2 after 6-7±3 h. At the end of the inoculum flask incubation, the inoculum culture is transferred into the bioreactor. The inoculum size is 2-4%. The fermentation conditions are controlled: pH 6.7 kept by addition of 25% NH₄₀H, 30° C., dissolved oxygen kept at 30%, air flow 15 to 30 standard litres per minute (SLPM), stir speed 50-800 rpm (cascade mode) until the final OD600 of 35-40 is reached. GMMA released into the fermentation broth are purified using two consecutive Tangential Flow Filtration (TFF) steps: a microfiltration in which the culture supernatant containing the GMMA is separated from the bacteria (eventually replaced by a centrifugation step), and an ultrafiltration, in which the GMMA are separated from soluble proteins [3].

### GMMA Characterization

Purified GMMA are characterized by dot blot for identity, total OAg content is quantified by HPAEC-PAD, total protein content by micro BCA and OAg/total protein ratio is calculated. Lipid A amount is quantified by HPLC-RP MS, while particle size is determined by Dynamic light scattering or HPLC-SEC/MALS. Protein pattern is qualitatively investigated by SDS PAGE analysis. Percentage of soluble proteins is determined by protein quantification in the supernatant after GMMA ultracentrifugation and residual DNA is quantified by threshold method.

The OAg is extracted after treatment with acetic acid and characterized for O-acetyl content by NMR or Hestrin colorimetric method. OAg molecular size distribution is measured by HPLC-SEC/semicarbazide and Lipid A structure is confirmed by MALDI-TOF.

OAg Identity by Dot Blot The OAg identity assay by dot blot method is designed to detect the presence of the respective OAg on the different GMMA DSs (Drug Substances). In this technique, the DS is applied directly on a membrane using a dot blotting apparatus. Then, the presence of the OAg is determined by immune-reaction. After blocking the membrane, a specific commercial antiserum or monoclonal antibody is used as primary antibody. The binding of the primary antibody is then detected using an enzyme labelled secondary antibody. The presence of immunoreacting OAg is detected by addition of substrate solution and formation of an insoluble purple dye.

As negative control, in the same assay, standard GMMA of the other three DSs are tested.

OAg Quantification by HPAEC-PAD (S. flexneri 1b, 2a, 3a)

Quantification of the OAg is performed by High-Performance Anion-Exchange Chromatography with Pulsed Amperometric Detection (HPAEC-PAD) analysis after acid hydrolysis of the sample. The quantification determines the concentration of Rhamnose present in the sample. A standard dilution series of a solution containing rhamnose, glucose and N-acetylglucosamine is run in each HPAEC-PAD analysis series and used as calibration curve to quantify the rhamnose and verify the presence of the glucose and N-acetylglucosamine in the sample.

To 450 μL of each dilution of the standard and of the sample, 150 μL of 8 M TFA are added. All vials are heated in parallel at 100° C. for 4 hours. Samples are then chilled at 2° C. to 8° C. for approximately 30 minutes, dried in a centrifugal evaporator, resuspended in 450 μL of water, filtered and analysed. HPAEC-PAD is performed with a Dionex ICS3000 (or 5000) equipped with a CarboPac PA10 column coupled with PA10 guard column. Separation is performed at 25° C. using the following conditions:
30 min, 18 mM NaOH, flow rate 0.6 mL/min (separation step)
10 min 500 mM NaOH, flow rate 1.6 mL/min (column washing step)
38 min 18 mM NaOH, flow rate 1.6 mL/min (column equilibration step)

The effluent is monitored using an electrochemical detector.

The quantification of OAg is performed on the basis of the known sugar ratios present in the OAg repeating units (Rha x3; GlcNAc; Glc) common to all three S. flexneri strains. Rhamnose is quantified as nmol/mL directly from the analysis and the OAg quantity is then calculated considering the molecular weight of the repeating unit and the rhamnose content in the repeating unit (OAg (μg/mL)=Rhamnose (μmol/mL)*267.94).

OAg Quantification by HPAEC-PAD (S. sonnei)

Quantification of the OAg in S. sonnei GMMA is performed by HPAEC-PAD analysis after acid hydrolysis of the sample. The quantification determines the concentration of amino-altruronic acid present in the sample. The OAg content as μg/mL is calculated considering the molecular weight of the repeating unit.

A dilution series of S. sonnei OAg standard 0.16-2.56 μg/mL is run in each HPAEC-PAD analysis and the areas of the resulting peaks are used to build a standard curve, interpolate the peak area of the unknown sample and quantify the corresponding μg/mL. S. sonnei GMMA express capsular polysaccharide and OAg, both sharing the same sugar dimer as repeating unit. Therefore, the result of the analysis is the sum of the two components.

To 300 μL of each dilution of the standard curve and of the sample, 1000 μL of a TFA/HCl mixture (1:6.7 v/v) are added. All vials are heated in parallel at 80° C. for 4.5 hours (final concentrations: HCl 8M TFA 10%). Samples are then chilled at 2° C. to 8° C. for approximately 15 minutes, dried for 2 hours under nitrogen flux and finally in a centrifugal evaporator, resuspended in 300 μL of water, filtered and analysed. HPAEC-PAD is performed with a Dionex ICS3000 (or 5000) equipped with a CarboPac PA1 column coupled with PA1 guard column. Separation is performed at 25° C. using the following condition:
15 min, 400 mM NaOH, flow rate 1.6 mL/min (no washing step needed between injections)

The effluent is monitored using an electrochemical detector.

### Protein Quantification by Micro BCA Assay

The total protein content is measured using the micro BCA assay (Micro BCA assay kit 23225, ThermoFischer scientific). This assay makes use of the protein reactivity that in alkaline conditions reduces copper (II) ions which are revealed through the formation of a purple complex with bicinchoninic acid. The amount of protein in the sample is determined by reading the 562 nm absorbance of the resulting product against a standard curve. The standard curve for the measurements is generated with bovine serum albumin (BSA) in the range of 1.8-20 μg/mL.

To quantify the soluble protein the assay is applied on the sample supernatant after GMMA removal by ultracentrifugation (110000 rpm, 30 minutes 4° C., using a rotor with K factor 15).

OAg O-Acetyl Content (S. flexneri 1b, 3a) by Hestrin-Dische

To measure the O-acetyl ester content of the S. flexneri OAg, the GMMA samples are treated by mild acid hydrolysis (2 h 100° C. 1% acetic acid) and the extracted OAg is then desalted using a disposable PD10 column (GE Healthcare).

On the same desalted extracted OAg sample, the O-acetylation using the Hestrin colorimetric method and the rhamnose content using the Dische colorimetric assay are measured. The O-acetyl content is then calculated as molar ratio between the O-acetyl ester and rhamnose content expressed as percentage.

The Hestrin method is based on reaction of the ester groups with hydroxylamine in a basic media to form hydroxamic acid which, at low pH, generates complexes with iron^{III} ions with maximum absorbance at 540 nm. Acetylcholine standard solution at several dilutions is treated as the sample in the assay and used as calibration curve.

The Dische assay, in the first step uses concentrated sulfuric acid at 100° C. on sample/standard that hydrolyses the OAg and allows the monosaccharides to form methyl furfural (from methyl pentoses) or hydroxymethyl furfural (from hexoses). Both these species react with sulfur present in cysteine added in the second step of the method resulting in chromophores. To have a signal coming only from methylpentose monomers (i.e. rhamnose), in the assay, the 396-427 nm abs difference is used. Fucose standard solution at several dilutions is treated as the sample in the assay and used as calibration curve (all methyl pentose monomers have the same color yield).

### OAg Molecular Size Distribution by HPLC-SEC Semicarbazide

In order to analyse the OAg chain length and structural integrity, the OAg is extracted from the GMMA by mild acid hydrolysis, which cleaves the labile linkage between lipid A and KDO residue at the end of the core region of the OAg chain. The OAg is released in the supernatant, separated from most of the lipids and proteins that precipitate in acidic conditions.

The HPLC-SEC of the extracted polysaccharides using a refractive index detector (column: TOSOH TSK G3000PW-XL column with guard column, flow rate 0.5 mL/mL, eluent: sodium phosphate 0.1M, NaCl 0.1M pH 7.2 with 5% acetonitrile) shows the molecular size distribution of the OAg chains present in the respective GMMA DS. The differential refractive index (dRI) can also evidence the presence of other polysaccharides like CPS (present in S. sonnei GMMA). Eventual residual DNA and glycogen polysaccharide are not separated by CPS peak in this analysis. (For S. sonnei, the relative amount of G4C-CPS can be determined with a further test, i.e. capsular polysaccharide quantification by HPAEC-PAD, after separation via Sephacryl S300 size exclusion chromatography.)

The molecular weight of the polysaccharide populations is assigned by gel permeation chromatography (GPC) analysis using standard dextran calibration curve.

In the same HPLC-SEC analysis, extracted OAg is also injected after derivatization of KDO with semicarbazide monitoring the 252 UV absorption. The molecular size distribution shown in such chromatograms is selectively related to OAg, with the peaks area proportional to the molar amount of OAg chains. In the same analysis KDO monomer standard solution at several dilutions is treated with semicarbazide, as the sample, and the peak areas are used as calibration curve for the OAg. This allows to measure relative ratio in moles of OAg populations at different average size. It should be noted that for S. sonnei GMMA the CPS peak is not labelled by semicarbazide.

### Protein Profile of Shigella GMMA by SDS-PAGE

The protein composition of GMMA is analysed by SDS-PAGE of GMMA after treatment in sample buffer containing SDS and 1,4-dithiothreitol (DTT). Proteins present in GMMA are dissociated into their polypeptide chains by treatment with the detergent sodium dodecyl sulfate (SDS) after reduction of the disulphide bonds using DTT and are then separated via SDS-PAGE. Bands are visualized with Coomassie blue staining and compared visually. Protein profiles obtained are compared with the profile of the reference standard GMMA electrophoresed in parallel.

### Lipid a Quantification by HPLC-RP MS

Quantitative determination of the lipid A in the GMMA is performed by quantifying selectively the 3-hydroxymyristic acid present as ester in the lipid A structure that contains two 3-hydroxymyristic esters. The quantification determines the number of moles of lipid A molecules that correspond to the half of the determined 3-hydroxymyristic acid. A standard dilution series of 3-hydroxymyristic acid is run in each HPLC-RP analysis series and used as calibration curve.

The procedure consists of an initial hydrolysis of the sample to release 3-hydroxyfatty acids cleaving quantitatively the ester bonds from the glucosamine O-3 present in the lipid-A.

The quantification is performed on HPLC-RP equipped with an ESI, triple quadrupole MS detector by SRM (or Thermo Orbitrap detector by PRM), with a calibration curve built on dilutions of the analyte standard, pre-treated as for GMMA sample hydrolysis. The first quadrupole of the detector is set to filter a mass corresponding to 3-hydroxymyristate anion (243 M/z), the collision cell is set to fragment the analyte structure and the third quadrupole is set to filter acetate anion (59 M/z). By this way the signal read from the detector is selectively due only to acetate anion produced by fragmentation of 3-hydroxymiristate anion.

Samples of the GMMA DS and the dilutions of the 3-hydroxymiristic standard are treated after dilution with isopropanol (alcohol final concentration 50%) with 250 mM sodium hydroxide (final concentration) for 2 h at 40° C. Samples are then chilled at 2° C. to 8° C. and analysed.

The chromatographic run is performed in isocratic conditions with 70% acetonitrile in water solution containing 0.05% formic acid with a flow rate of 250 μL/min on a C8 column (Phenomenex, Luna 3u C8 50×2 mm).

Prior chromatographic separation, the sample/standard injected in HPLC is subjected to an on-line SPE extraction step (Phenomenex Strata-X 20×2 mm On-line extraction cartridge) using methanol 40% in water containing 0.05% formic acid as solvent for loading and washing from salts and matrix components.

### In Vitro Characterization of Residual Endotoxic Effect of Genetically Modified LPS

The removal of the msbB gene homologues was verified at the MCB (Master Cell Bank) level by PCR. Inactivation of such genes ensures lipid A modification to a non-WT form with no possibility of reversion to a WT hexa-acylated form.

For additional characterization, a direct analysis of the lipid A by Matrix-Assisted Laser Desorption/Ionization—time of flight (MALDI-TOF) and the Monocyte-Activation test (MAT) are carried out at GVGH. Both tests provide confirmation of the lipid A genetic modification.

### Analysis of the Lipid a by MALDI-TOF

MALDI-TOF analysis is performed to determine the lipid A structure in LPS molecules of Shigella GMMA. This test provides a confirmation of the genetic lipid A modification.

For the assay, the lipid A is separated after treatment of GMMA with acetic acid and then assayed by MALDI.

GMMA with a protein concentration of about 1 mg/mL (micro BCA calibration curve) or a cell bank suspension with an OD600 of about 3 (4 mL sample) are treated with 1% acetic acid (final concentration) for 2 or 6 hours, respectively, at 100° C. to obtain a precipitate containing the lipid A. The precipitate is then collected, washed with water and the lipid A is extracted in chloroform/methanol 4:1. The final solution, which contains the lipid A, is mixed 1:1 with Super DHB (Fluka, 50862) saturated solution (acetonitrile/water 1:1). Two microliters of the mixture are loaded onto the target plate and after the spot is dried at room temperature, the plate is inserted in the mass spectrometer.

The spectra (negative reflectron mode) generally show peaks corresponding to the lipid A molecular species and contain several peaks due to fragmentation of the lipid A (i.e. loss of one or more fatty acid chains), sodium adduct (+22 m/z) and lipid A dephosphorylation (−80 m/z). The species of lipid A is identified by comparison of the molecular peak mass m/z to what is expected for the sample in analysis.

### Vaccine Formulation

The altSonflex 1-2-3 vaccine is formulated to contain 15 μg OAg of each of the four Drug Substance components (i.e. 15 μg OAg from GMMA from each of S. sonnei NVGH2929, S. flexneri 1b NVGH2858, S. flexneri 2a NVGH2404 and S. flexneri 3a NVGH2766) (60 μg OAg in total) in a 0.5 mL dose.

The formulation is aseptically performed by sequential transfer of sterile water for injection (quantity depends on concentration of aluminium hydroxide batch), GMMA suspension with 600 μg/mL total OAg to reach in the bulk a final concentration of 120 μg/mL OAg (30 μg/mL OAg of each component), aluminium hydroxide to reach in the bulk a final concentration of 0.7 mg aluminium per mL, sterile sodium phosphate 100 mM pH 6.5 to reach a final concentration of 10 mM and NaCl 1540 mM to reach a final concentration of 154 mM.

The formulation procedure was developed at small scale and scaled up to 3 L to produce altSonflex 1-2-3 adsorbed on aluminium hydroxide GMP lots. In more detail, the laboratory formulation processes consist of:
1. Thawing and pooling all the Drug Substance aliquots.
2. Dilution of the mixed Drug Substance suspension with saline to reach a final concentration of 600 μg OAg/mL (150 μg/mL of each OAg Drug Substance), and subsequent 0.22 μm filtration with Sartobran P150 filter.
3. Adsorption step:
   i. Dilution of the GMMA with WFI under constant stirring 180 rpm to reach a final concentration of 120 μg OAg/mL (30 μg/mL of each OA Drug Substance) in the final formulated bulk.
   ii. Addition of aluminium hydroxide to reach a final concentration of 0.7 mg Al³⁺/mL in the final formulated bulk, followed by continuous stirring at 180 rpm for 2 hours. Note that before adding the aluminium hydroxide, the aluminium hydroxide suspension is kept under constant stirring at 150-200 rpm for 30±10 minutes, after a stirrer has been added under the laminar flow hood.
4. Addition of 100 mM sodium phosphate buffer pH 6.5 to reach 10 mM in the final formulated bulk followed by continuous stirring at 180 rpm for 15 minutes.
5. Addition of NaCl solution 1540 mM to reach 154 mM in the final formulated bulk followed by continuous stirring at 180 rpm for 15 h+4 h.

### Example 3. Determination of Immunogenicity and Serum Bactericidal Assay (SBA) Titres

### Immunogenicity Studies

To assess the immunogenicity of the altSonflex 1-2-3 vaccine, groups of 8 female CD1 mice were immunized intraperitoneally with 5 different doses of the vaccine (0.58 ng, 2.3 ng, 9.4 ng, 37 ng and 150 ng of each OAg in 0.2 mL). All doses are prepared by dilutions with diluent composed of Alhydrogel in 10 mM sodium phosphate buffer pH 6.5 and 154 mM sodium chloride (0.7 mg Al³⁺/mL). Eight mice per group are immunized twice at days 0 and 28, blood is collected at day 27 and 42 and the serum obtained. IgG antibodies elicited to S. sonnei LPS and S. flexneri 1b, 2a and 3a OAg are assessed by ELISA. ELISA plates are coated with LPS/OAg (LPS for S. sonnei, OAg for S. flexneri), blocked with PBS milk 5%, and incubated with the sera diluted 1:100, 1:4,000 and 1:160,000 in PBS-Tween 0.05%. Bound antibodies are then detected using an enzyme-labelled secondary antibody (anti-mouse IgG-alkaline phosphatase) in PBS-Tween 0.05%. The presence of immunoreacting anti-S. sonnei LPS/S. flexneri 1b/2a/3a OAg IgG is detected by addition of substrate solution and formation of a yellow colour detected by absorbance at 405 nm subtracted by the absorbance at 490 nm. The samples are tested in comparison to calibrated mouse anti-S. sonnei LPS/S. flexneri 1b/2a/3a OAg reference standard sera. Results are expressed in ELISA units/mL determined relative to the reference serum. One ELISA unit equals the reciprocal of the dilution of the reference serum that yields an OD of 1 in the assay. The dose-response curves obtained from the sera after immunization with the freshly formulated immunogenicity reference standard and with the test batch are compared using statistical methods.

A similar study was done in rabbits, where altSonflex 1-2-3 was compared to corresponding monovalent formulations at 1.5 μg each OAg in 0.5 mL per dose. New Zealand female rabbits 8/group were immunized intramuscularly at days 0 and 28. Blood is collected at days 27 and 42 and the serum obtained.

As shown in FIGS. 10 and 11, the altSonflex 1-2-3 comprising S. sonnei GMMA and S. flexneri 1b, 2a and 3a GMMA was found to be highly immunogenic in mice and rabbits, and there was no evidence of antigenic interference.

### Serum Bactericidal Assay as a Measure of Antibody Functionality

Single sera were also tested against wild-type bacterial strains in serum bactericidal assay (SBA) based on a luminescent readout [13; 9]. Shigella sonnei virG::cat, S. flexneri 1b, 2a and 3a bacteria working cell banks, stored frozen at −80° C. in 20% glycerol stock, was grown overnight (16-18 hours) at 37° C. in LB medium (supplemented with 20 μg/mL of chloramphenicol in case of S. sonnei), with stirring at 180 rpm. The overnight bacterial suspension was then diluted in fresh LB medium to OD600 nm of 0.05 and incubated at 37° C. with 180 rpm agitation in an orbital shaker until it reached an OD600 of 0.2+/−0.02. SBA was performed in 96-well round-bottom sterile plates (Corning, New York, NY, USA) by incubating serial dilutions in PBS of heat-inactivated (HI) test sera in the presence of exogenous baby rabbit complement (BRC) and bacteria for 3 hours at 37° C. Log-phase cultures prepared as described above were diluted in PBS or LB and added in the reaction well to an approximate concentration of 1×10⁵ colony forming unit (CFU)/assay well. BRC at the final concentration of 20% for S. sonnei, 15% for S. flexneri 1b and 3a and 7.5% for S. flexneri 2a respectively was present in the reaction mixtures. For each serum dilution curve, a control well with no HI serum was added. Wells containing standard sera were also added to the plate to validate the assay. At the end of the incubation, the SBA plate was centrifuged at RT for 10 min at 4000×g. The supernatant was discarded to remove ATP derived from dead bacteria and SBA reagents; the remaining live bacterial pellets were resuspended in PBS, transferred in a white round-bottom 96-well plate (Greiner, Kremsmünster, Austria) and mixed 1:1 v:v with BacTiter-Glo Reagent (Promega, Madison, MA, USA). The reaction was incubated for 5 min at RT on an orbital shaker, and the luminescence signal measured by a luminometer (Viktor, Perkin Elmer, Waltham, MA, USA). A 4-parameter non-linear regression was applied to raw luminescence data obtained for all sera dilutions tested; an arbitrary serum dilution of 10¹⁵ was assigned to the control well containing no sera. Fitting was performed by weighting the data for the inverse of luminescence squared. Results of the assay were expressed as the IC50, the reciprocal serum dilution that resulted in a 50% reduction of luminescence and thus corresponding to 50% growth inhibition of the bacteria present in the assay. GraphPad Prism 7 software was used for curve fitting and IC50 determination. Titers below the minimum measurable signal were assigned a titer corresponding to half of the first dilution of sera tested.

As shown in FIGS. 10 and 12, the altSonflex 1-2-3 comprising S. sonnei GMMA and S. flexneri 1b, 2a and 3a GMMA was able to elicit functional antibodies in mice and rabbits, and there was no evidence of antigenic interference.

### Example 4. Increasing the Dose of O-Antigen Increases the Immune Response to the altSonflex 1-2-3 Vaccine

The immunogenicity and SBA experiments described in Example 3 were also carried out for different doses of O-antigen within the GMMA from each Shigella strain, namely S. sonnei, and S. flexneri 1b, 2a and 3a.

As shown in FIGS. 13 and 14, the altSonflex 1-2-3 vaccine is able to induce anti-LPS/OAg IgG in mice in a O-antigen dose responsive manner. The vaccine is also able to induce functional antibodies in a O-antigen dose responsive manner (FIGS. 15 and 16). This was established in both mice and rabbits, and was verified through Spearman rank correlation (alpha=0.05).

### Example 5. Higher O-Antigen Doses Used in altSonflex 1-2-3 are not Reactogenic

### Pyrogenicity Tests

The inventors established a modification of the European Pharmacopeia intravenous pyrogenicity test method (Ph.Eur. 2.6.8 pyrogens) using the administration of a full human dose delivered intramuscularly. Groups of 3 rabbits (naïve female New Zealand White Rabbits, 10 to 11 weeks old at start of study, Weight≥1.5 kg), preselected according Ph.Eur. 2.6.8 pyrogens, were placed in retaining boxes and the body temperatures were recorded using a rectal probe and the initial temperature was determined. The lot of the vaccine to be tested (0.5 mL) was injected intramuscularly to each of three rabbits of the vaccine group and 0.5 mL sterile physiological saline to the three rabbits of the control group. Automated temperature recording from rectal probe by software was done until 3 hours after administration. Subsequent temperature read outs from rectal probe at 210, 240, 270 and 300 minutes were recorded manually. Following the last temperature measurement on day 0, rabbits were removed from restraining cages. Temperatures were again recorded on day 1, 24 hours after injection. At 6, 24 and 48 hours after immunization, rabbits were observed for signs of good health and injection site abnormalities. At the end of 48 hours observation period, rabbits were removed from study and euthanized. The animals were euthanized by injection of Dolethal intra cardiac (400 mg/kg). The maximum temperature rise for each rabbit is determined (the difference between the highest temperature measured during the 5 h period after administration and the initial temperature). For the test to be valid, the mean of the maximum temperature rise of the three controls has to be <0.3° C.

The first clinical experience with a vaccine based on the GMMA technology, the Shigella sonnei 1790GAHB vaccine, revealed an acceptable safety profile in clinical studies, in which 192 adult participants received the vaccine at different dose levels. During three Phase 1 and two Phase 2 trials only one participant had short lasting elevated temperature above 38° C. for one day after vaccination, even though rabbits developed mean maximum temperature rises of 0.4 and 0.5° C. after batch testing with the modified pyrogenicity test using intramuscular administration of the highest full human dose (Table 4). The temperature rises in the modified pyrogenicity test after administration of altSonflex 1-2-3 vaccine were higher but short lasting as with the 1790GAHB vaccine (Table 5). The increase in temperature of the individual rabbits did not exceed 41° C. and was observed to decrease in 24 hours or less (FIG. 17).

TABLE 4
Composition of altSonflex1-2-3 and 4-component GMMA mix
tested in the modified rabbit pyrogenicity test

| | altSonflex1-2-3 | altSonflex1-2-3 |
|---|---|---|
| | (clinical batch | (toxicology batch |
| | AL-21-001) | LBP-5819) |
| Total OAg, μg | 60 | 60 |
| Protein, μg | 106.3 | 89.5 |
| Lipid A, nmol | 32.3 | 27.6 |
| Aluminium, mg | 0.35 | 0.35 |

TABLE 5
Results of pyrogenicity test in rabbits after intramuscular
administration of altSonflex1-2-3 vaccine

| | | | Group mean | Time of | |
|---|---|---|---|---|---|
| | | | temp. [° C.] | mean | |
| | Group | Group | at end of | peak | Mean |
| Administered | average | mean | measurement | temp. | peak |
| vaccines, | initial | maximum | [5 hours | after | temp. |
| (doses based | temp. | temp. | after | vaccination | rise |
| on OAg) | [° C.] | [° C.] | vaccination] | [minutes] | [° C.] |
| altSonflex1-2-3 | 38.8 | 40.2 | 39.4 | 180 | 1.4 |
| vaccine | | | | | |
| (toxicology batch | | | | | |
| LBP-5819) | | | | | |
| altSonflex1-2-3 | 38.4 | 40.1 | 39.0 | 180 | 1.7 |
| vaccine | | | | | |
| (clinical batch | | | | | |
| AL-21-001) | | | | | |

### Monocyte Activation Tests (MATs)

The Monocyte Activation Test is performed following Ph. Eur. Chapter 2.6.30 Method C: Reference lot comparison test. The test is run on at least 4 individual PBMC donors or using PBMC from four pooled donors.

Peripheral Blood Mononuclear Cells (PBMC) are purified from buffy coats of healthy donors by a density gradient of Ficoll-Plaque Plus. Working aliquots containing around 50 millions PBMC/each are frozen and stored in liquid nitrogen until use. Lots are “validated” using endotoxin standard (LPS from E. coli) prior use also to determine the optimal stimulation range.

Adequate amount of working PBMC aliquots are freshly cultured at a density of 125,000 cells in 250 μL of RPMI 1640 medium complemented with 25 mM HEPES, 2 mM glutamine, 2% Human Serum (HS), 1% penicillin/streptomycin (complete RPMI) in 96-well round bottom plates. Nine 2-fold serial dilutions GMMA Drug Product test lots and reference GMMA lot are diluted in complete RPMI, and assayed in the optimal stimulation range depending on the PBMC donor used (usually 0.006-10 ng/ml final concentration in the assay). Stimuli are added in four replicates, including control wells stimulated with aluminium hydroxide only and endotoxin standards. Cells are incubated for 20 h at 37° C., and supernatants are recovered after centrifugation of the plates at 400 g.

IL6 released in the supernatants are measured by sandwich ELISA. Briefly Nunc MaxiSorp 96-well plates are coated overnight at 4° C. with 2 g/ml human IL-6 capture antibody in PBS, subsequently washed three times with PBS with 0.05% Tween 20 (PBST), blocked for 1 h with PBS with 1% BSA at room temperature, and washed three times with PBST. Supernatants from PBMC are added and incubated for 2 h at room temperature. A 2-fold dilution series of recombinant human IL-6 of 31.24 to 4,000 μg/ml was included in duplicate as standard curve on each plate. Plates were washed three times with PBST. Bound IL-6 is detected using 2 μg/ml biotin-conjugated anti-human IL-6 in PBST with 0.1% BSA for 2 h at room temperature, followed by three washes with PBST, 20 min of incubation at room temperature with streptavidin/horseradish peroxidase diluted 1:200 in PBST with 0.1% BSA, three washes with PBST, and a colour reaction with 100 μl/well substrate for 8 min at room temperature in the dark. The reaction is stopped by adding 50 μl/well of 12.5% sulfuric acid. The plates are read at 450 and 540 nm and the A450-540 nm is determined. IL-6 concentrations in the samples are calculated in comparison with the standard.

For the analysis of the cytokine release by PBMC, the average cytokine levels (or A450-540 nm) in the assays are plotted against the GMMA concentration (or dilution factor). Data analysis is performed comparing GMMA Drug Product test lot/s to reference lots (1790GAHB in the example above) using CombiStats or IL6 concentration at 10-fold increase in cytokine release over background. The optimal lot identified as reference is represented by the mixture of the 4 corresponding Drug Substances that ensures good parallelism with the Drug Product lot independently from the donor used. In this case, the Relative Pyrogenic Unit (RPU) of the samples and the Geometric Coefficient of Variation (GCV) percentage are calculated against the reference lot by statistical means as defined in [Carson, 2021]. As the intrinsic biological variability of the assay is about 2-fold, only RPU values of >2 are considered to indicate difference of pyrogenic activity.

Use of the altSonflex 1-2-3 vaccine has been tested in MAT and shown to induce similar proinflammatory cytokine release as 1790GAHB, namely the vaccine described in Example 1, when compared by starting dilutions from corresponding full human doses (FIG. 18).

### Example 6-Clinical Trial Protocol for Testing altSonflex 1-2-3 Vaccine in Humans

A staged Phase I/II observer-blind, randomised, controlled, multi-country study is being carried out to evaluate the safety, reactogenicity, and immune responses to the altSonflex 1-2-3 vaccine in humans. The study has been carried out in adults in Europe (Stage 1) and will be followed by age de-escalation from adults to children and infants, and dose-finding in infants in Africa (Stage 2).

Different doses of O-antigen of GMMA from each of S. sonnei, and S. flexneri 1b, 2a and 3a will be used, specifically 3.75 μg (dose A), 7.5 μg (dose B) and 15 μg (dose C) per dose. An aluminium-based adjuvant will be used, preferably aluminium hydroxide, at the same dose for each of the three antigen doses.

The following dosage regimens will be used:

| | | No of | Timing | Shigella vaccine (SV)/ | |
|---|---|---|---|---|---|
| Age group | Territory | doses | of doses | placebo (P)/other | Dose |
| 18 to 50 | Europe | 2 | Days 1 and 85 | SV or P | C |
| years | | | | | |
| 18 to 50 | Europe | 2 | Days 1 and 169 | SV or P | C |
| years | | | | | |
| 18 to 50 | Africa | 2 | Days 1 and 85 | Menveo at day 1 and | |
| years | | | | Boostrix at day 85 | |
| 18 to 50 | Africa | 2 | Days 1 and 85 | SV | C |
| years | | | | | |
| 24 to 59 | Africa | 2 | Days 1 and 85 | Menveo at day 1 and | |
| months | | | | Typhim Vi at day 85 | |
| | | | | (control group for children | |
| | | | | receiving SV Dose B in | |
| | | | | row below) | |
| 24 to 59 | Africa | 2 | Days 1 and 85 | SV | B |
| months | | | | | |
| 24 to 59 | Africa | 2 | Days 1 and 85 | Menveo at day 1 and | |
| months | | | | Typhim Vi at day 85 | |
| | | | | (control group for children | |
| | | | | receiving SV Dose C in | |
| | | | | row below) | |
| 24 to 59 | Africa | 2 | Days 1 and 85 | SV | C |
| months | | | | | |
| 9 months | Africa | N/A | N/A | Menveo at day 1 and day | |
| | | | | 85, Infanrix IPV at day 253 | |
| | | | | and two doses of MR-Vac | |
| | | | | at days 29 and 281 (control | |
| | | | | group for infants receiving | |
| | | | | SV Dose A in row below) | |
| 9 months | Africa | 3 | Days 1, 85, | SV at days 1, 85 and 253, | A |
| | | | and 253 | and MR-Vac at days 29 | |
| | | | | and 281 | |
| 9 months | Africa | N/A | N/A | Menveo at day 1 and day | |
| | | | | 85, Infanrix IPV at day 253 | |
| | | | | and two doses of MR-Vac | |
| | | | | at days 29 and 281 (control | |
| | | | | group for infants receiving | |
| | | | | SV Dose B in row below) | |
| 9 months | Africa | 3 | Days 1, 85, | SV at days 1, 85 and 253, | B |
| | | | and 253 | and MR-Vac at days 29 | |
| | | | | and 281 | |
| 9 months | Africa | N/A | N/A | Menveo at day 1 and day | |
| | | | | 85, Infanrix IPV at day 253 | |
| | | | | and two doses of MR-Vac | |
| | | | | at days 29 and 281 (control | |
| | | | | group for infants receiving | |
| | | | | SV Dose C in row below) | |
| 9 months | Africa | 3 | Days 1, 85, | SV at days 1, 85 and 253, | C |
| | | | and 253 | and MR-Vac at days 29 | |
| | | | | and 281 | |
| 9 months | Africa | N/A | N/A | Menveo at day 1 and day | |
| | | | | 85, Infanrix IPV at day 253 | |
| | | | | and two doses of MR-Vac | |
| | | | | at days 29 and 253 (control | |
| | | | | group for infants receiving | |
| | | | | SV Doses A, B, C in rows | |
| | | | | below) | |
| 9 months | Africa | 3 | Days 1, 85, | SV at days 1, 85 and 253, | A |
| | | | and 253 | and MR-Vac at days 29 | |
| | | | | and 253 | |
| 9 months | Africa | 3 | Days 1, 85, | SV at days 1, 85 and 253, | B |
| | | | and 253 | and MR-Vac at days 29 | |
| | | | | and 253 | |
| 9 months | Africa | 3 | Days 1, 85, | SV at days 1, 85 and 253, | C |
| | | | and 253 | and MR-Vac at days 29 | |
| | | | | and 253 | |

FIGS. 19 and 20 show the results of the clinical trial carried out in Europe, specifically after one dose (15 μg) of the altSonflex 1-2-3 vaccine has been given.

In order to measure serum bactericidal activity post-vaccination, anti-S. sonnei and S. flexneri 1b, 2a and 3a LPS serum IgG was measured at Day 1 and 28 days post-vaccination. For each group, geometric mean concentrations (GMCs) and their 95% CIs were computed by exponentiating (base 10) the mean and 95% CIs of the log 10 ELISA concentrations. Bactericidal activity was assessed by a luminescent serum bactericidal assay (L-SBA) [9][14] at Day 1 and 28 days post-vaccination. SBA geometric mean titres (GMTs) were tabulated with their 95% CIs. Antibody concentrations below the lower limit of quantification (LLOQ) (22 EU/mL for ELISA and inhibition concentration [IC]50 of 33.5 for L-SBA) were set to half that limit for the purpose of analysis. Within-subject geometric mean ratios (GMR) were computed for GMC/GMT at each post-vaccination time points versus baseline levels and by exponentiating the mean within-subject differences in log-transformed titres and the corresponding CIs.

All statistical analyses were carried out using Statistical Analysis Systems 9.4.

FIG. 19 demonstrates that immunisation with altSonflex 1-2-3 elicits antibodies with stronger bactericidal activity against S. sonnei compared to those seen in previous studies with 1790GAHB (as described in Example 1). FIG. 19 shows that more patients immunised with altSonflex 1-2-3 had a bactericidal activity 4-fold higher than baseline and within-subject GMR significantly increased respect to previous trials with 1790GAHB.

FIG. 20 demonstrates that altSonflex 1-2-3 also elicited good levels of bactericidal antibody titers against each of S. flexneri strains 2a, 3a and 1b.

The trial also found that the altSonflex 1-2-3 vaccine was non-reactogenic. In summary:
The most frequently reported solicited Administration-site event was pain, experienced by about 79% of participants
There was no report of severe fever among trial participants after receiving at least one dose of the study intervention
At least 1 Unsolicited AE was experienced by 68% of participants, most of them were administration site and general disorders
Medically attended AEs were experienced by 13 participants after dose 1 reporting 15 events
   All events were not related to study vaccination by the INV
   All events were mild to moderate in severity except for 2 severe events (1 cervicitis and 1 influenza like illness)
   For all the events, medical assistance was provided by Medical Personnel (except for 1 event of severe neck pain that was assisted at the Emergency room).

No SAE occurred during the study (No AEs leading to Death or leading to hospitalization)
No grade 3 deviations in laboratory parameters from baseline or deviations from normal
10 Participants withdrawn from the study, none related to Adverse Events

### REFERENCES

1. Formal S B, Kent T H, May H C, Palmer A, Falkow S, LaBrec E H. Protection of monkeys against experimental shigellosis with a living attenuated oral polyvalent dysentery vaccine. J Bacteriol. 1966; 92:17-22
2. Makino S, Sasakawa C, Kamata K, Kurata T, Yoshikawa M. A genetic determinant required for continuous reinfection of adjacent cells on large plasmid in S. flexneri 2a. Cell. 1986; 46:551-555
3. Berlanda Scorza F, Colucci A M, Maggiore L, Sanzone S, Rossi O, Ferlenghi I, et al. High yield production process for Shigella outer membrane particles. PLOS One. 2012; 7: e35616
4. Clementz T, Bednarski J J, Raetz C R. Function of the htrB high temperature requirement gene of Escherichia coli in the acylation of lipid A. J Biol Chem. 1996; 271:12095-12102.
5. Rossi O, Pesce I, Giannelli C, Aprea S, Caboni M, Citiulo F, et al. Modulation of Endotoxicity of Shigella Generalized Modules for Membrane Antigens (GMMA) by Genetic Lipid A Modifications: Relative Activation of TLR4 and TLR2 Pathways in Different Mutants. J Biol Chem. 2014; 289:24922-24935
6. White S J, Freedman L S. Allocation of patients to treatment groups in a controlled clinical study. Br J Cancer 1978; 37 (5): 849-57
7. Launay O, Lewis D J M, Anemona A, et al. Safety profile and immunologic responses of a novel vaccine against Shigella sonnei administered intramuscularly, intradermally and intranasally: Results from two parallel randomized phase 1 clinical studies in healthy adult volunteers in Europe. E Bio Medicine 2017; 22:164-72
8. Cohen D, Block C, Green M S, Lowell G, Ofek I. Immunoglobulin M, A, and G antibody response to lipopolysaccharide O antigen in symptomatic and asymptomatic Shigella infections. J Clin Microbiol 1989; 27 (1): 162-7
9. Rossi O, Molesti E, Saul A, Giannelli C, Micoli F, Necchi F. Intra-laboratory evaluation of luminescence based high-throughput serum bactericidal assay (L-SBA) to determine bactericidal activity of human sera against Shigella. High Throughput 2020; 9 (2): 14)
10. Gerke C, Colucci A M, Giannelli C, Sanzone S, Vitali C G, Sollai L, et al. (2015) Production of a Shigella sonnei Vaccine Based on Generalized Modules for Membrane Antigens (GMMA), 1790GAHB. PLOS ONE 10 (8): e0134478.
11. Mancini, F., Gasperini, G., Rossi, O. et al. Dissecting the contribution of O-Antigen and proteins to the immunogenicity of Shigella sonnei generalized modules for membrane antigens (GMMA). Sci Rep 11, 906 (2021).
12. Datsenko K A, Wanner B L. One-step inactivation of chromosomal genes in Escherichia coli K-12 using PCR products. Proc Natl Acad Sci USA. 2000 Jun. 6; 97 (12): 6640-5. doi: 10.1073/pnas. 120163297. PMID: 10829079; PMCID: PMC18686.
13. Necchi F, Saul A, Rondini S (2017) Development of a high-throughput method to evaluate serum bactericidal activity using bacterial ATP measurement as survival readout. PLOS ONE 12 (2): e0172163. https://doi.org/10.1371/journal.pone.0172163
14. Mancini, F.; Micoli, F.; Rossi, O. (2022) Setup and Characterization of a High-Throughput Luminescence-Based Serum Bactericidal Assay (L-SBA) to Determine Functionality of Human Sera against Shigella flexneri. BioTech 11, no. 3:29. https://doi.org/10.3390/biotech11030029

### Further Aspects of the Invention

1. An immunogenic composition comprising:
(a) Shigella sonnei GMMA; and
(b) Shigella flexneri GMMA,
wherein each of the GMMA comprises modified lipid A, and the immunogenic composition comprises at least 10 μg of O-antigen.

2. The immunogenic composition of aspect 1, wherein the immunogenic composition is a single dose.

3. The immunogenic composition of aspect 1 or 2, wherein the Shigella flexneri GMMA comprises Shigella flexneri serotype 1b GMMA.

4. The immunogenic composition of aspect 3, wherein the Shigella flexneri serotype 1b GMMA comprises Shigella flexneri serotype 1b O-antigen.

5. The immunogenic composition of aspect 3 or 4, wherein the Shigella flexneri serotype 1b GMMA is derived from Shigella flexneri serotype 1b bacteria.

6. The immunogenic composition of any one of aspects 3 to 5, wherein the amount of O-antigen in the Shigella flexneri serotype 1b GMMA is at least 10, 11, 12, 13, 14 or 15 μg.

7. The immunogenic composition of any one of aspects 3 to 6, wherein the amount of O-antigen in the Shigella flexneri serotype 1b GMMA is between 10 and 25 μg.

8. The immunogenic composition of any one of aspects 3 to 7, wherein the amount of O-antigen in the Shigella flexneri serotype 1b GMMA is between 12 and 20 μg.

9. The immunogenic composition of any one of aspects 3 to 8, wherein the amount of O-antigen in the Shigella flexneri serotype 1b GMMA is about 15 μg.

10. The immunogenic composition of any one of the preceding aspects, wherein the Shigella flexneri GMMA comprises Shigella flexneri serotype 2a GMMA.

11. The immunogenic composition of aspect 10, wherein the Shigella flexneri serotype 2a GMMA comprises Shigella flexneri serotype 2a O-antigen.

12. The immunogenic composition of aspect 10 or 11, wherein the Shigella flexneri serotype 2a GMMA is derived from Shigella flexneri serotype 2a bacteria.

13. The immunogenic composition of any one of aspects 10 to 12, wherein the amount of O-antigen in the Shigella flexneri serotype 2a GMMA is at least 10, 11, 12, 13, 14 or 15 μg.

14. The immunogenic composition of any one of aspects 10 to 13, wherein the amount of O-antigen in the Shigella flexneri serotype 2a GMMA is between 10 and 25 μg.

15. The immunogenic composition of any one of aspects 10 to 14, wherein the amount of O-antigen in the Shigella flexneri serotype 2a GMMA is between 12 and 20 μg.

16. The immunogenic composition of any one of aspects 10 to 15, wherein the amount of O-antigen in the Shigella flexneri serotype 2a GMMA is about 15 μg.

17. The immunogenic composition of any one of the preceding aspects, wherein the Shigella flexneri GMMA comprises Shigella flexneri serotype 3a GMMA.

18. The immunogenic composition of aspect 17, wherein the Shigella flexneri serotype 3a GMMA comprises Shigella flexneri serotype 3a O-antigen.

19. The immunogenic composition of aspect 17 or 18, wherein the Shigella flexneri serotype 3a GMMA is derived from Shigella flexneri serotype 3a bacteria.

20. The immunogenic composition of any one of aspects 17 to 19, wherein the amount of O-antigen in the Shigella flexneri serotype 3a GMMA is at least 10, 11, 12, 13, 14 or 15 μg.

21. The immunogenic composition of any one of aspects 17 to 20, wherein the amount of O-antigen in the Shigella flexneri serotype 3a GMMA is between 10 and 25 μg.

22. The immunogenic composition of any one of aspects 17 to 21, wherein the amount of O-antigen in the Shigella flexneri serotype 3a GMMA is between 12 and 20 g.

23. The immunogenic composition of any one of aspects 17 to 22, wherein the amount of O-antigen in the Shigella flexneri serotype 3a GMMA is about 15 μg.

24. The immunogenic composition of any one of the preceding aspects, wherein the Shigella flexneri GMMA comprises Shigella flexneri serotype 6 GMMA.

25. The immunogenic composition of aspect 24, wherein the Shigella flexneri serotype 6 GMMA comprises Shigella flexneri serotype 6 O-antigen.

26. The immunogenic composition of aspect 24 or 25, wherein the Shigella flexneri serotype 6 GMMA is derived from Shigella flexneri serotype 6 bacteria.

27. The immunogenic composition of any one of aspects 24 to 26, wherein the amount of O-antigen in the Shigella flexneri serotype 6 GMMA is at least 10, 11, 12, 13, 14 or 15 μg.

28. The immunogenic composition of any one of aspects 24 to 27, wherein the amount of O-antigen in the Shigella flexneri serotype 6 GMMA is between 10 and 25 μg.

29. The immunogenic composition of any one of aspects 24 to 28, wherein the amount of O-antigen in the Shigella flexneri serotype 6 GMMA is between 12 and 20 μg.

30. The immunogenic composition of any one of aspects 24 to 29, wherein the amount of O-antigen in the Shigella flexneri serotype 6 GMMA is about 15 μg.

31. The immunogenic composition of any one of the preceding aspects, wherein the Shigella flexneri GMMA comprises GMMA from at least two Shigella flexneri serotypes from serotypes 1b, 2a, 3a and 6.

32. The immunogenic composition of any one of the preceding aspects, wherein the Shigella flexneri GMMA comprises GMMA from Shigella flexneri serotypes 1b, 2a, and 3a.

33. The immunogenic composition of any one of the preceding aspects, wherein the Shigella flexneri GMMA comprises GMMA from each of Shigella flexneri serotypes 1b, 2a, 3a and 6.

34. The immunogenic composition of any one of aspects 31 to 33, wherein the amount of O-antigen in each of the Shigella flexneri serotype 1b, 2a, and 3a GMMA is at least 14 μg.

35. The immunogenic composition of any one of the preceding aspects, wherein the amount of O-antigen in the Shigella sonnei GMMA is at least 10, 11, 12, 13, 14 or 15 μg.

s

36. The immunogenic composition of any one of the preceding aspects, wherein the amount of O-antigen in the Shigella sonnei GMMA is between 10 and 25 μg.

37. The immunogenic composition of any one of the preceding aspects, wherein the amount of O-antigen in the Shigella sonnei GMMA is between 12 and 20 μg.

38. The immunogenic composition of any one of the preceding aspects, wherein the amount of O-antigen in the Shigella sonnei GMMA is about 15 μg. mono

39. The immunogenic composition of any one of the preceding aspects, wherein the total amount of O-antigen in the Shigella sonnei GMMA and Shigella flexneri GMMA is at least 30, 35, 40, 45, 50, 55 or 60 μg.

40. The immunogenic composition of any one of the preceding aspects, wherein the total amount of O-antigen in the Shigella sonnei GMMA and Shigella flexneri GMMA is between 40 and 100 μg.

41. The immunogenic composition of any one of the preceding aspects, wherein the total amount of O-antigen in the Shigella sonnei GMMA and Shigella flexneri GMMA is between 50 and 70 μg.

42. The immunogenic composition of any one of the preceding aspects, wherein the total amount of O-antigen in the Shigella sonnei GMMA and Shigella flexneri GMMA is about 60 μg.

43. The immunogenic composition of any one of the preceding aspects, wherein the Shigella sonnei GMMA are derived from Shigella sonnei that is ΔmsbB1 and/or the ΔmsbB2.

44. The immunogenic composition of any one of the preceding aspects, wherein the Shigella flexneri GMMA are derived from Shigella flexneri that is ΔmsbB1 and/or ΔmsbB2.

45. The immunogenic composition of any one of the preceding aspects, wherein the modified lipid A is modified such that it is less toxic than the corresponding wildtype lipid A.

46. The immunogenic composition of any one of the preceding aspects, wherein the modified lipid A is a penta-acylated lipid A.

47. The immunogenic composition of any one of the preceding aspects, wherein the modified lipid A is a lipid A in which C14 comprises a myristoyl group.

48. The immunogenic composition of any one of the preceding aspects, which comprises an adjuvant.

49. The immunogenic composition of aspect 48, wherein the adjuvant is an aluminium adjuvant.

50. The immunogenic composition of aspect 48 or 49, wherein the adjuvant is aluminium phosphate or aluminium hydroxide.

51. The immunogenic composition of any one of aspects 48 to 50, wherein the adjuvant is aluminium hydroxide.

52. The immunogenic composition of any one of the preceding aspects, which is formulated in a pre-filled syringe.

53. The immunogenic composition of any one of the preceding aspects, which is suitable for treatment of a human patient.

54. The immunogenic composition of any one of the preceding aspects, which comprises at least one pharmaceutical carrier(s) and/or excipients.

55. The immunogenic composition of aspect 54, which is a pharmaceutical or vaccine composition.

56. The immunogenic composition of any one of aspects 1 to 55, wherein, on administration to a subject, the immunogenic composition elicits an increased number of antibodies with anti-Shigella sonnei and/or Shigella flexneri bactericidal activity compared to a composition that comprises a lower amount of O-antigen from corresponding Shigella sonnei and/or Shigella flexneri.

57. The immunogenic composition of aspect 56, which comprises at least two times, three times, four times, five times, six times, seven times, eight times, nine times, or ten times as much O-antigen compared to the composition that comprises a lower amount of O-antigen from corresponding Shigella sonnei and/or Shigella flexneri.

58. The immunogenic composition of aspect 56, which comprises up to and including two times, three times, four times, five times, six times, seven times, eight times, nine times, or ten times as much O-antigen compared to the composition that comprises a lower amount of O-antigen from corresponding Shigella sonnei and/or Shigella flexneri.

59. The immunogenic composition of any one of aspects 56 to 58, wherein the immunogenic composition is suitably immunogenic such that, after immunisation with the immunogenic composition, a higher number of patients have at least a 4-fold increase in bactericidal activity compared with patients immunised with the composition that comprises a lower amount of O-antigen from corresponding Shigella sonnei and/or Shigella flexneri.

60. A method of increasing the number of antibodies with anti-Shigella sonnei and/or Shigella flexneri bactericidal activity in a subject, comprising administering the immunogenic composition of any one of aspects 1 to 59 to the subject.

61. Use of the immunogenic composition of any one of aspects 1 to 59 in the manufacture of a medicament for use in a method of increasing the number of antibodies with anti-Shigella sonnei and/or Shigella flexneri bactericidal activity in a subject.

62. The immunogenic composition of any one of aspects 1 to 59 for use in a method of increasing the number of antibodies with anti-Shigella sonnei and/or Shigella flexneri bactericidal activity in a subject.

63. The immunogenic composition of any one of aspects 1 to 59 for use in a method of preventing or treating infection by Shigella in a subject.

64. A method of preventing or treating infection by Shigella in a subject comprising administering the immunogenic composition of any one of aspects 1 to 59 to a subject.

65. Use of the immunogenic composition of any one of aspects 1 to 59 in the manufacture of a medicament for use in a method of preventing or treating infection by Shigella in a subject.

66. The immunogenic composition for use of aspect 62 or 63 or the use of aspect 61 or 65, wherein the method comprises administering the immunogenic composition of any one of aspects 1 to 59 to the subject.

67. The immunogenic composition for use of aspect 62 or 63, method of aspect 60 or 64, or the use of aspect 61 or 65, wherein the method comprises administering a first dose and a second dose to the subject and the first dose and/or the second dose comprises the immunogenic composition of any one of aspects 1 to 59.

68. The immunogenic composition for use of aspect 62 or 63, the method of aspect 60 or 64, or the use of aspect 61 or 65, wherein the method comprises administering a first dose, a second dose, and a third dose to the subject and the first dose, the second dose and/or the third dose comprises the immunogenic composition of any one of aspects 1 to 59.

69. The immunogenic composition for use, method, or use of aspect 67 or 68, wherein the first dose is administered at day one.

70. The immunogenic composition for use, method, or use of any one of aspects 67 to 69, wherein the second dose is administered between day 60 and 100 or between day 80 and 90.

71. The immunogenic composition for use, method, or use of any one of aspects 67 to 70, wherein the second dose is administered around day 85.

72. The immunogenic composition for use, method, or use of any one of aspects 67 to 69, wherein the second dose is administered between day 120 and 250 or between day 160 and 180.

73. The immunogenic composition for use, method, or use of any one of aspects 67 to 69 or 72, wherein the second dose is administered around day 169.

74. The immunogenic composition for use, method, or use of any one of aspects 67 to 69, 72 or 73, wherein the third dose is administered between day 200 and 300 or between day 245 and 260.

75. The immunogenic composition for use, method, or use of any one of aspects 67 to 69, or 72 to 74, wherein the third dose is administered around day 253.

76. The immunogenic composition for use, method, or use of any one of aspects 63 to 75, wherein the method for preventing or treating infection by Shigella comprises administering a second, different, immunogenic composition.

77. The immunogenic composition for use, method, or use of aspect 76, wherein the second, different, immunogenic composition comprises live, attenuated, measles virus and/or live, attenuated, rubella virus.

78. The immunogenic composition for use, method, or use of aspect 77, wherein the live, attenuated, measles virus is derived from the Edmonston strain and/or the live, attenuated, rubella virus is derived from the Wistar RA 27/3 strain.

79. The immunogenic composition for use, method, or use of aspect 78, wherein two unit doses of the second, different, immunogenic composition are administered on separate days.

80. The immunogenic composition for use, method, or use of aspect 79, wherein the first unit dose of the second, different, immunogenic composition is administered between day 10 and 50.

81. The immunogenic composition for use, method, or use of aspects 79 and 80, wherein the first unit dose of the second, different, immunogenic composition is administered around day 29.

82. The immunogenic composition for use, method, or use of aspects 79 to 81, wherein the second unit dose of the second, different, immunogenic composition is administered between day 250 and 350.

83. The immunogenic composition for use, method, or use of aspects 79 to 82, wherein the second unit dose of the second, different, immunogenic composition is administered around day 281.

84. The immunogenic composition for use of aspect 62 or 63, method of aspect 60 or 64, or the use of aspect 61 or 65, wherein the subject is human.

85. The immunogenic composition for use, method, or use of aspect 84, wherein the subject is 18 years old or above 18 years old.

86. The immunogenic composition for use, method, or use of aspect 84, wherein the subject is below 18 years old.

87. The immunogenic composition for use, method, or use of aspect 84 or 86, wherein the subject is between 12 to 72 months.

88. The immunogenic composition for use, method, or use of aspects 84, 86 or 87, wherein the subject is between 24 to 59 months.

89. The immunogenic composition for use, method, or use of aspect 84 or 86, wherein the subject is between 6 to 12 months.

90. The immunogenic composition for use, method, or use of aspect 84, 86 or 89, wherein the subject is around 9 months.

91. The immunogenic composition for use of aspect 63, method of aspect 64, the use of aspect 65, the immunogenic composition for use or use of aspect 66, or the immunogenic composition for use, method, or use of aspects 67 to 90, wherein the infection by Shigella comprises infection by Shigella sonnei.

92. The immunogenic composition for use of aspect 63, method of aspect 64, the use of aspect 65, the immunogenic composition for use or use of aspect 66, or the immunogenic composition for use, method, or use of aspects 67 to 91, wherein the infection by Shigella comprises infection by Shigella flexneri 1b.

93. The immunogenic composition for use of aspect 63, method of aspect 64, the use of aspect 65, the immunogenic composition for use or use of aspect 66, or the immunogenic composition for use, method, or use of aspects 67 to 92, wherein the infection by Shigella comprises infection by Shigella flexneri 2a.

94. The immunogenic composition for use of aspect 63, method of aspect 64, the use of aspect 65, the immunogenic composition for use or use of aspect 66, or the immunogenic composition for use, method, or use of aspects 67 to 93, wherein the infection by Shigella comprises infection by Shigella flexneri 3a.

95. The immunogenic composition for use of aspect 62, method of aspect 60, or the use of aspect 61, wherein the Shigella flexneri is Shigella flexneri 1b, 2a and/or 3b.

1. An immunogenic composition comprising:
(a) Shigella sonnei GMMA; and
(b) Shigella flexneri GMMA,
wherein each of the GMMA comprises modified lipid A, and the immunogenic composition comprises at least 10 μg of O-antigen.

2. The immunogenic composition of claim 1, wherein the immunogenic composition is a single dose.

3. The immunogenic composition of claim 1 or 2, wherein the Shigella flexneri GMMA comprises Shigella flexneri serotype 1b GMMA.

4. The immunogenic composition of claim 3, wherein the Shigella flexneri serotype 1b GMMA comprises Shigella flexneri serotype 1b O-antigen.

5. The immunogenic composition of claim 3 or 4, wherein the Shigella flexneri serotype 1b GMMA is derived from Shigella flexneri serotype 1b bacteria.

6. The immunogenic composition of any one of claims 3 to 5, wherein the amount of O-antigen in the Shigella flexneri serotype 1b GMMA is at least 10, 11, 12, 13, 14 or 15 μg.

7. The immunogenic composition of any one of the preceding claims, wherein the Shigella flexneri GMMA comprises Shigella flexneri serotype 2a GMMA.

8. The immunogenic composition of claim 7, wherein the Shigella flexneri serotype 2a GMMA comprises Shigella flexneri serotype 2a O-antigen.

9. The immunogenic composition of claim 7 or 8, wherein the Shigella flexneri serotype 2a GMMA is derived from Shigella flexneri serotype 2a bacteria.

10. The immunogenic composition of any one of claims 7 to 9, wherein the amount of O-antigen in the Shigella flexneri serotype 2a GMMA is at least 10, 11, 12, 13, 14 or 15 μg.

11. The immunogenic composition of any one of the preceding claims, wherein the Shigella flexneri GMMA comprises Shigella flexneri serotype 3a GMMA.

12. The immunogenic composition of claim 11, wherein the Shigella flexneri serotype 3a GMMA comprises Shigella flexneri serotype 3a O-antigen.

13. The immunogenic composition of claim 11 or 12, wherein the Shigella flexneri serotype 3a GMMA is derived from Shigella flexneri serotype 3a bacteria.

14. The immunogenic composition of any one of claims 11 to 13, wherein the amount of O-antigen in the Shigella flexneri serotype 3a GMMA is at least 10, 11, 12, 13, 14 or 15 μg.

15. The immunogenic composition of any one of the preceding claims, wherein the amount of O-antigen in each of the Shigella flexneri serotype 1b, 2a, and 3a GMMA is at least 14 μg.

16. The immunogenic composition of any one of the preceding claims, wherein the amount of O-antigen in the Shigella sonnei GMMA is at least 10, 11, 12, 13, 14 or 15 μg.

17. The immunogenic composition of any one of the preceding claims, wherein the total amount of O-antigen in the Shigella sonnei GMMA and Shigella flexneri GMMA is at least 30, 35, 40, 45, 50, 55 or 60 μg.

18. The immunogenic composition of any one of the preceding claims, wherein the Shigella sonnei GMMA are derived from Shigella sonnei that is ΔmsbB1 and/or ΔmsbB2.

19. The immunogenic composition of any one of the preceding claims, wherein the Shigella flexneri GMMA are derived from Shigella flexneri that is ΔmsbB1 and/or ΔmsbB2.

20. The immunogenic composition of any one of the preceding claims, wherein the modified lipid A is modified such that it is less toxic than the corresponding wildtype lipid A.

21. The immunogenic composition of any one of the preceding claims, wherein the modified lipid A is a penta-acylated lipid A.

22. The immunogenic composition of any one of the preceding claims, wherein the modified lipid A is a lipid A in which C14 comprises a myristoyl group.

23. The immunogenic composition of any one of the preceding claims, which comprises an adjuvant.

24. The immunogenic composition of claim 23, wherein the adjuvant is an aluminium adjuvant.

25. The immunogenic composition of any one of claims 1 to 24 for use in a method of preventing or treating infection by Shigella in a subject.
